(19) United States
(12) Patent Application Publication
(10) Pub. No.: US 2010/0163034 A1
(43) Pub. Date: Jul. 1, 2010

(54) Methods and systems for presenting an inhalation experience

(75) Inventors: Roderick A. Hyde, Redmond, WA (US); Robert Langer, Newton, MA (US); Eric C. Leuthardt, St. Louis, MO (US); Robert W. Lord, Seattle, WA (US); Elizabeth A. Sweeney, Seattle, WA (US); Clarence T. Tegreene, Bellevue, WA (US); Lowell L. Wood, JR., Bellevue, WA (US)

Correspondence Address: IV - SUITER SWANTZ PC LLO, 14301 FNB PARKWAY , SUITE 220, OMAHA, NE, 68154, US

(73) Assignee: Searete LLC, a limited liability Corporation of the State of Delaware

(21) Appl. No.: 12/380,108

(22) Filed: Feb. 23, 2009

Related U.S. Application Data
(63) Continuation-in-part of application No. 12/317,934, filed on Dec. 30, 2008.
Application No. 12/317,934 is a continuation-in-part of application No. 12/319,143, filed on Dec. 31, 2008.
Application No. 12/319,143 is a continuation-in-part of application No. 12/378,284, filed on Feb. 12, 2009.
Application No. 12/378,284 is a continuation-in-part of application No. 12/378,485, filed on Feb. 13, 2009.
Application No. 12/378,485 is a continuation-in-part of application No. 12/380,013, filed on Feb. 20, 2009.

(51) Int. Cl. A61M 15/00; G01N 33/497; G05B 13/02; G06F 17/00

(52) U.S. Cl. 128/203.14; 73/23.3; 700/28; 700/94; 700/90

(57) ABSTRACT

Methods, computer program products, and systems are described that include detecting in an exhalant at least one indication of bioactive agent use by an individual and altering at least one artificial sensory experience at least partly based on the at least one indication of bioactive agent use by an individual.

### CROSS-REFERENCE TO RELATED APPLICATIONS

The present application is related to and claims the benefit of the earliest available effective filing date(s) from the following listed application(s) (the “Related Applications”) (e.g., claims earliest available priority dates for other than provisional patent applications or claims benefits under 35 USC § 119(e) for provisional patent applications, for any and all parent, grandparent, great-grandparent, etc. applications of the Related Application(s)).

### RELATED APPLICATIONS

For purposes of the USPTO extra-statutory requirements, the present application constitutes a continuation-in-part of U.S. patent application entitled METHODS AND SYSTEMS FOR PRESENTING AN INHALATION EXPERIENCE, naming RODERICK A. HYDE; ROBERT LANGER; ERIC C. LEUTHARDT; ELIZABETH A. SWEENEY; CLARENCE T. TEGREENE; AND LOWELL L. WOOD as inventors, filed Dec. 30, 2008, application Ser. No. 12/319,143, which is currently co-pending, or is an application of which a currently co-pending application is entitled to the benefit of the filing date.
For purposes of the USPTO extra-statutory requirements, the present application constitutes a continuation-in-part of U.S. patent application entitled METHODS AND SYSTEMS FOR PRESENTING AN INHALATION EXPERIENCE, naming RODERICK A. HYDE; ROBERT LANGER; ERIC C. LEUTHARDT; ELIZABETH A. SWEENEY; CLARENCE T. TEGREENE; AND LOWELL L. WOOD as inventors, filed Dec. 31, 2008, application Ser. No. 12/317,934, which is currently co-pending, or is an application of which a currently co-pending application is entitled to the benefit of the filing date.
For purposes of the USPTO extra-statutory requirements, the present application constitutes a continuation-in-part of U.S. patent application entitled METHODS AND SYSTEMS FOR PRESENTING AN INHALATION EXPERIENCE, naming RODERICK A. HYDE; ROBERT LANGER; ERIC C. LEUTHARDT; ELIZABETH A. SWEENEY; CLARENCE T. TEGREENE; AND LOWELL L. WOOD as inventors, filed Feb. 12, 2009, application Ser. No. NOT YET ASSIGNED, which is currently co-pending, or is an application of which a currently co-pending application is entitled to the benefit of the filing date.
For purposes of the USPTO extra-statutory requirements, the present application constitutes a continuation-in-part of U.S. patent application entitled METHODS AND SYSTEMS FOR PRESENTING AN INHALATION EXPERIENCE, naming RODERICK A. HYDE; ROBERT LANGER; ERIC C. LEUTHARDT; ELIZABETH A. SWEENEY; CLARENCE T. TEGREENE; AND LOWELL L. WOOD as inventors, filed Feb. 13, 2009, application Ser. No. NOT YET ASSIGNED, which is currently co-pending, or is an application of which a currently co-pending application is entitled to the benefit of the filing date.

The United States Patent Office (USPTO) has published a notice to the effect that the USPTO's computer programs require that patent applicants reference both a serial number and indicate whether an application is a continuation or continuation-in-part. Stephen G. Kunin, Benefit of Prior-Filed Application, USPTO Official Gazette Mar. 18, 2003, available at http://www.uspto.pov/web/offices/com/sol/op/2003/week11/patbene.htm. The present Applicant Entity (hereinafter “Applicant”) has provided above a specific reference to the application(s) from which priority is being claimed as recited by statute. Applicant understands that the statute is unambiguous in its specific reference language and does not require either a serial number or any characterization, such as “continuation” or “continuation-in-part,” for claiming priority to U.S. patent applications. Notwithstanding the foregoing, Applicant understands that the USPTO's computer programs have certain data entry requirements, and hence Applicant is designating the present application as a continuation-in-part of its parent applications as set forth above, but expressly points out that such designations are not to be construed in any way as any type of commentary and/or admission as to whether or not the present application contains any new matter in addition to the matter of its parent application(s).

All subject matter of the Related Applications and of any and all parent, grandparent, great-grandparent, etc. applications of the Related applications is incorporated herein by reference to the extent such subject matter is not inconsistent herewith.

### TECHNICAL FIELD

This description relates to methods and systems for an inhaled bioactive agent combined with an artificial sensory experience.

### SUMMARY

In one aspect, a method includes but is not limited to detecting in an exhalant at least one indication of bioactive agent use by an individual and altering at least one artificial sensory experience at least partly based on the at least one indication of bioactive agent use by an individual. In addition to the foregoing, other method aspects are described in the claims, drawings, and text forming a part of the present disclosure.

In one or more various aspects, related systems include but are not limited to circuitry and/or programming for effecting the herein-referenced method aspects; the circuitry and/or programming can be virtually any combination of hardware, software, and/or firmware configured to effect the herein-referenced method aspects depending upon the design choices of the system designer.

In one aspect, a system includes but is not limited to means for detecting in an exhalant at least one indication of bioactive agent use by an individual and means for altering at least one artificial sensory experience at least partly based on the at least one indication of bioactive agent use by an individual. In addition to the foregoing, other method aspects are described in the claims, drawings, and text forming a part of the present disclosure.

In one aspect, a system includes but is not limited to circuitry for detecting in an exhalant at least one indication of bioactive agent use by an individual and circuitry for altering at least one artificial sensory experience at least partly based on the at least one indication of bioactive agent use by an individual. In addition to the foregoing, other method aspects are described in the claims, drawings, and text forming a part of the present disclosure.

In one aspect, a computer program product includes but is not limited to a signal-bearing medium bearing one or more instructions for detecting in an exhalant at least one indication of bioactive agent use by an individual and one or more instructions for altering at least one artificial sensory experience at least partly based on the at least one indication of bioactive agent use by an individual. In addition to the foregoing, other method aspects are described in the claims, drawings, and text forming a part of the present disclosure.

In one aspect, a system includes but is not limited to a computing device and instructions that when executed on the computing device cause the computing device to detect in an exhalant at least one indication of bioactive agent use by an individual and alter at least one artificial sensory experience at least partly based on the at least one indication of bioactive agent use by an individual. In addition to the foregoing, other method aspects are described in the claims, drawings, and text forming a part of the present disclosure.

The foregoing is a summary and thus may contain simplifications, generalizations, inclusions, and/or omissions of detail; consequently, those skilled in the art will appreciate that the summary is illustrative only and is NOT intended to be in any way limiting. Other aspects, features, and advantages of the devices and/or processes and/or other subject matter described herein will become apparent in the teachings set forth herein.

### BRIEF DESCRIPTION OF THE FIGURES

FIG. 1 illustrates an exemplary environment in which one or more technologies may be implemented.

FIG. 2 illustrates an exemplary environment in which one or more technologies may be implemented.

FIG. 3 illustrates an exemplary inhalation device.

FIG. 4 illustrates an exemplary environment in which one or more technologies may be implemented.

FIG. 5 illustrates an exemplary environment in which one or more technologies may be implemented.

FIG. 6 illustrates an operational flow representing example operations related to combining an inhaled bioactive agent and an artificial sensory experience.

FIG. 7 illustrates an alternative embodiment of the operational flow of FIG. 6.

FIG. 8 illustrates an alternative embodiment of the operational flow of FIG. 6.

FIG. 9 illustrates an alternative embodiment of the operational flow of FIG. 6.

FIG. 10 illustrates an alternative embodiment of the operational flow of FIG. 6.

FIG. 11 illustrates an alternative embodiment of the operational flow of FIG. 6.

FIG. 12 illustrates an alternative embodiment of the operational flow of FIG. 6.

FIG. 13 illustrates an alternative embodiment of the operational flow of FIG. 6.

FIG. 14 illustrates an alternative embodiment of the operational flow of FIG. 6.

FIG. 15 illustrates an alternative embodiment of the operational flow of FIG. 6.

FIG. 16 illustrates a computer program product related to combining an inhaled bioactive agent and an artificial sensory experience.

FIG. 17 illustrates a system related to combining an inhaled bioactive agent and an artificial sensory experience.

FIG. 18 illustrates an exemplary environment in which one or more technologies may be implemented.

FIG. 19 illustrates an exemplary environment in which one or more technologies may be implemented.

FIG. 20 illustrates an exemplary environment in which one or more technologies may be implemented.

FIG. 21 illustrates an exemplary environment in which one or more technologies may be implemented.

FIG. 22 illustrates an operational flow representing example operations related to combining an inhaled bioactive agent and an artificial sensory experience.

FIG. 23 illustrates an alternative embodiment of the operational flow of FIG. 22.

FIG. 24 illustrates an alternative embodiment of the operational flow of FIG. 22.

FIG. 25 illustrates an alternative embodiment of the operational flow of FIG. 22.

FIG. 26 illustrates an alternative embodiment of the operational flow of FIG. 22.

FIG. 27 illustrates an alternative embodiment of the operational flow of FIG. 22.

FIG. 28 illustrates an alternative embodiment of the operational flow of FIG. 22.

FIG. 29 illustrates an alternative embodiment of the operational flow of FIG. 22.

FIG. 30 illustrates an alternative embodiment of the operational flow of FIG. 22.

FIG. 31 illustrates an alternative embodiment of the operational flow of FIG. 22.

FIG. 32 illustrates an alternative embodiment of the operational flow of FIG. 22.

FIG. 33 illustrates an alternative embodiment of the operational flow of FIG. 22.

FIG. 34 illustrates a computer program product related to combining an inhaled bioactive agent and an artificial sensory experience.

FIG. 35 illustrates a system related to combining an inhaled bioactive agent and an artificial sensory experience.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

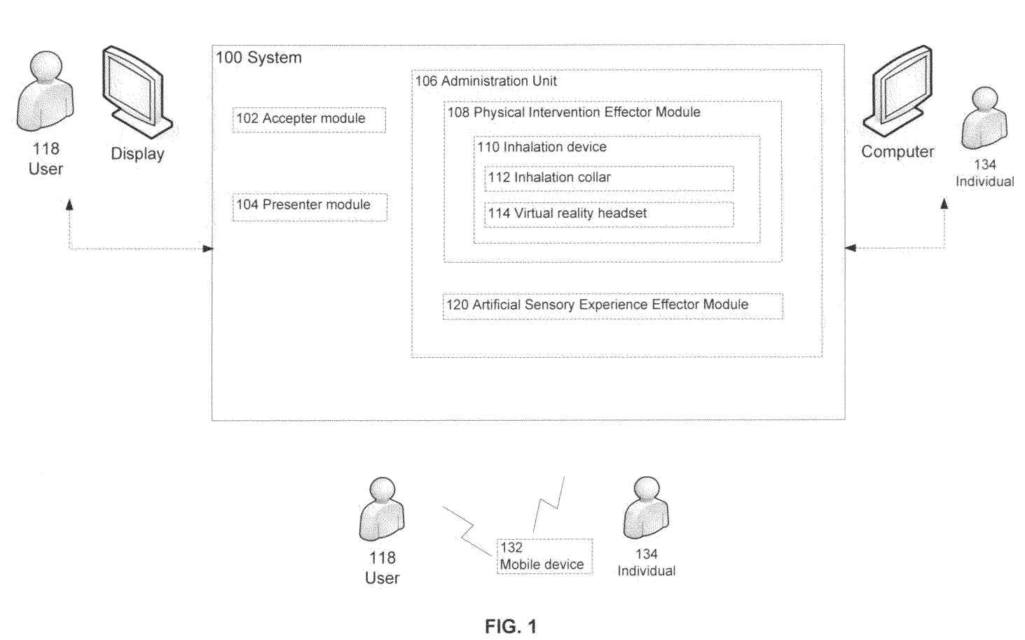

FIG. 1 illustrates system 100 for accepting an indication of at least one health-related condition and/or presenting an indication of at least one artificial sensory experience and an indication of at least one inhalation therapy at least partially based on the accepting at least one indication of a health-related condition. The system 100 may include accepter module 102, presenter module 104, and/or administration unit 106. Administration unit 106 may include physical intervention effector module 108 and/or artificial sensory experience effector module 120. Physical intervention effector module 108 may include inhalation device 110. Inhalation device 110 may include inhalation collar 112 and/or virtual reality headset 114. Additionally, system 3200 may include mobile device 132.

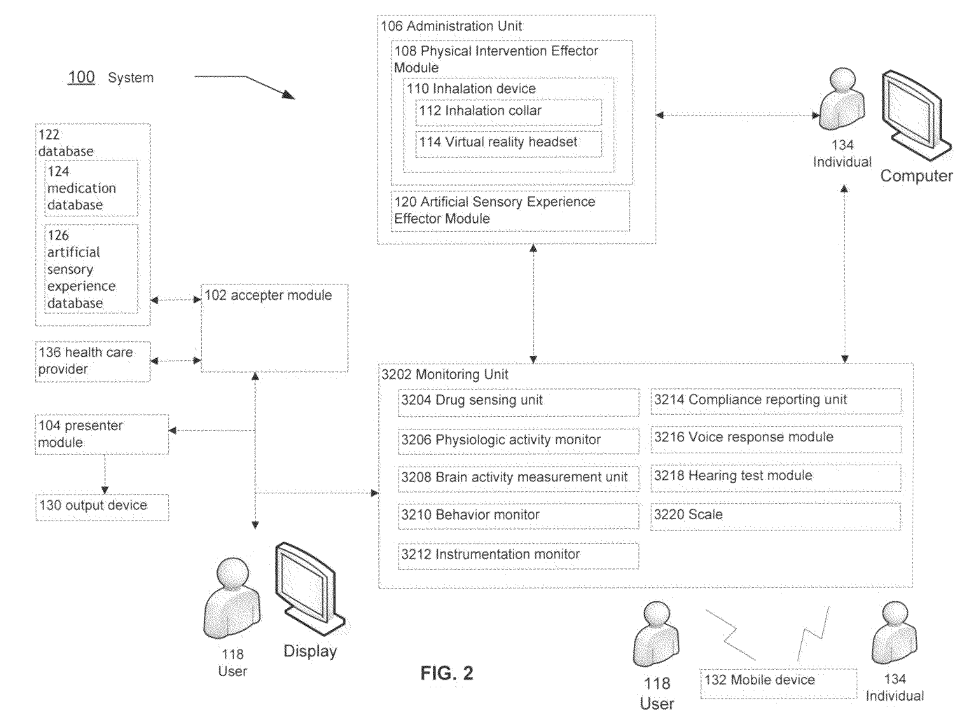

FIG. 2 illustrates system 100 for accepting an indication of at least one health-related condition and/or presenting an indication of at least one artificial sensory experience and an indication of at least one inhalation therapy at least partially based on the accepting at least one indication of a health-related condition. The system 100 may include accepter module 102, presenter module 104, administration unit 106, and/or monitoring unit 3202. Accepter module 102 may receive and/or transmit information and/or data to and/or from user 118, database 122, presenter module 3410, output device 130, and/or health care provider 136. Database 122 may include medication database 124 and/or artificial sensory experience database 126. Monitoring unit 3202 may monitor individual 134 and may include drug sensing unit 3204, physiologic activity monitor 3206, brain activity measurement unit 3208, behavior monitor 3210, instrumentation monitor 3212, compliance reporting unit 3214, voice response module 3216, hearing test module 3218, and/or scale 3220. Administration unit 106 may include physical intervention effector module 108 and/or artificial sensory experience effector module 120. Physical intervention effector module 108 may include inhalation device 110. Inhalation device 110 may include inhalation collar 112 and/or virtual reality headset 114. Additionally, mobile device 132 may communicate with accepter module 102, presenter module 104, healthcare provider 136, user 118, individual 134, monitoring unit 3202, and/or administration unit 3222.

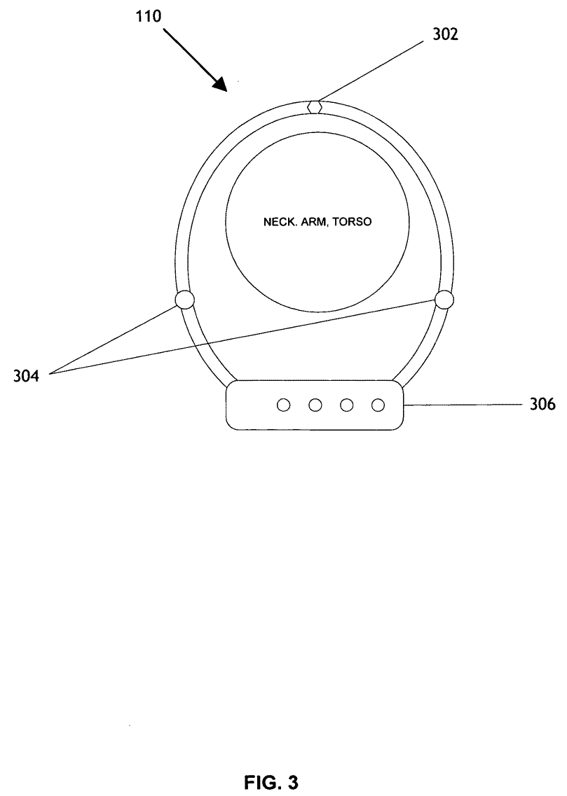

FIG. 3 illustrates an exemplary inhalation device 110. An exemplary inhalation device 110 may include a closure device, a transducer, and/or a dispensing reservoir. Inhalation device 110 may include, for example, a collar, a necklace, and/or a bracelet. Inhalation device 110 may include tubing, a chain, a polymer, a metal, a textile, and may be solid and/or hollow. Closure device 302 may include a buckle, Vetcro, a snap, a clasp, a lock, a coupler, elastic, and/or magnets. Transducer 304 may include a blood glucose monitor, a blood oxygen monitor, means for sending a signal to a reservoir to dispense medication, such as an antenna, means for powering the unit, such as a battery, memory, and/or a computer processor. Dispensing reservoir 306 may include means for power, such as a battery, means for receiving conditional input, such as a processor and/or memory, means for dispensing a bioactive agent in aerosol, dust and/or vapor form, such as a nebulizer, a sprayer, and/or a nozzle. Additionally, the dispensing reservoir 306 may be removable and/or refillable.

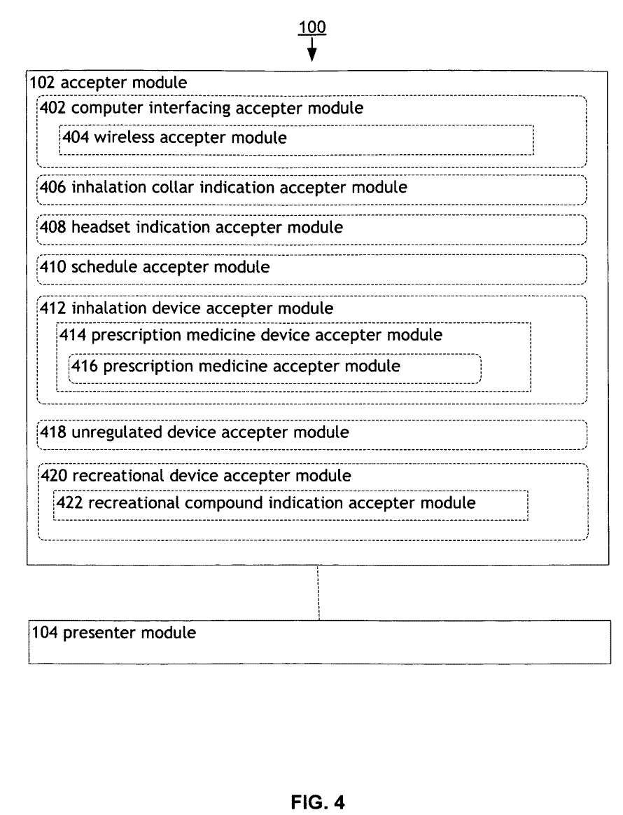

FIG. 4 further illustrates system 100 including accepter module 102 and/or presenter module 104. Accepter module 102 may include computer interfacing accepter module 402, inhalation collar indication accepter module 406, headset indication accepter module 408, schedule accepter module 410, inhalation device accepter module 412, unregulated device accepter module 418, and/or recreational device accepter module 420. Computer interfacing accepter module 402 may include wireless accepter module 404. Inhalation device accepter module 412 may include prescription medicine device accepter module 414 and/or prescription medicine accepter module 416. Recreational device accepter module 420 may include recreational compound indication accepter module 422.

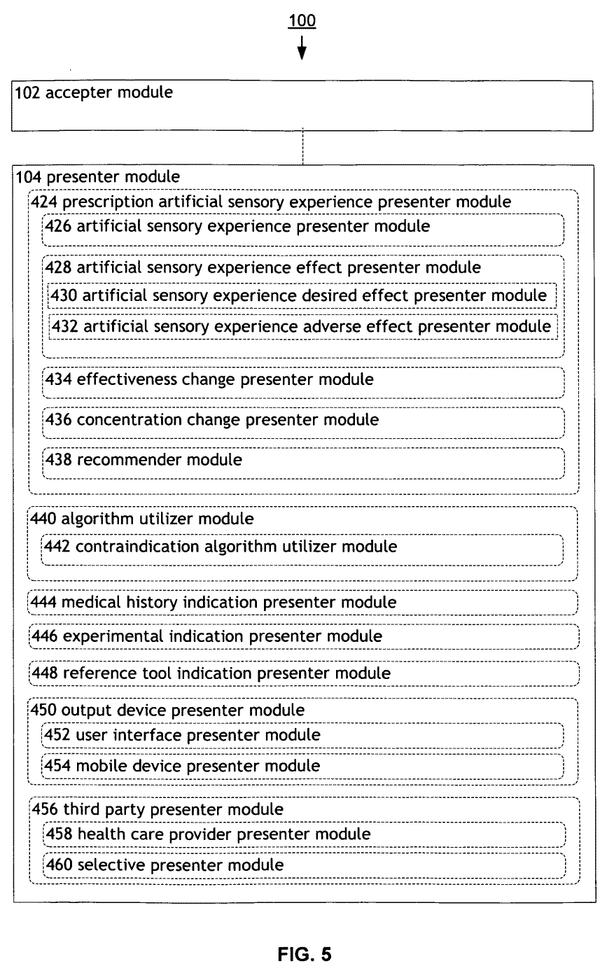

FIG. 5 illustrates system 100 including accepter module 102 and/or presenter module 104. Presenter module 104 may include prescription artificial sensory experience presenter module 424, algorithm utilizer module 440, medical history indication presenter module 444, experimental indiciation presenter module 446, reference tool indication presenter module 448, output device presenter module 450, and/or third party presenter module 456. Prescription artificial sensory experience presenter module 424 may include artificial sensory experience presenter module 426, artificial sensory experience effect presenter module 428, effectiveness change presenter module 434, concentration change presenter module 436, and/or recommender module 438. Artificial sensory experience effect presenter module 428 may include artificial sensory experience desired effect presenter module 430 and/or artificial sensory experience adverse effect presenter module 432. Algorithm utilizer module 440 may include contraindication algorithm utilizer module 442. Output device presenter module 450 may include user interface presenter module 452 and/or mobile device presenter module 454. Third party presenter module 456 may include health care provider presenter module 458 and/or selective presenter module 460.

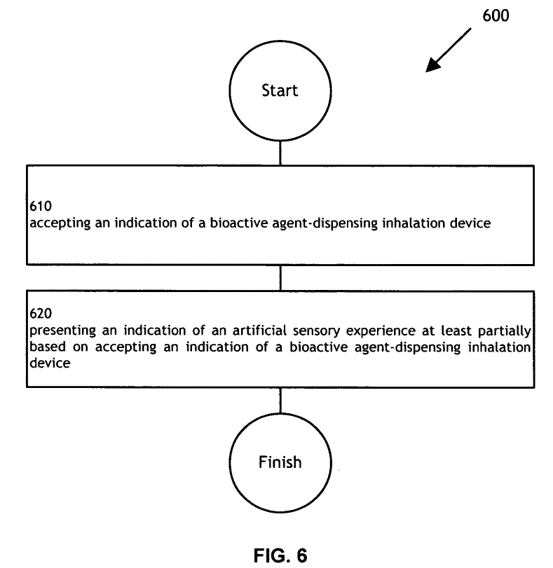

FIG. 6 illustrates an operational flow 600 representing example operations related to accepting an indication of at least one health-related condition and presenting an indication of at least one artificial sensory experience and an indication of at least one inhalation therapy at least partially based on the accepting at least one indication of a health-related condition. In FIG. 6 and in following figures that include various examples of operational flows, discussion and explanation may be provided with respect to the above-described examples of FIGS. 1 through 5, and/or with respect to other examples and contexts. However, it should be understood that the operational flows may be executed in a number of other environments and contexts, and/or in modified versions of FIGS. 1 through 5. Also, although the various operational flows are presented in the sequence(s) illustrated, it should be understood that the various operations may be performed in other orders than those which are illustrated, or may be performed concurrently.

After a start operation, the operational flow 600 moves to operation 610. Operation 610 depicts accepting an indication of at least one health-related condition. For example, as shown in FIGS. 1 through 5, accepter module 102 may accept an indication of a bioactive agent-dispensing inhalation device. One example of a bioactive agent-dispensing inhalation device may include an inhaler used for delivering a bioactive agent into the body using a body airway. Some other examples may include a collar, necklace, and/or a bracelet with a bioactive agent dispenser proximate to the nose, mouth, and/or inhalation route. In one embodiment, accepter module 102 may accept an indication of a bioactive agent-dispensing collar for dispensing a medication, such as a steroid and/or a bronchodilator. In some instances, accepter module 102 may include a computer processor, a user interface, and/or computer memory.

Then, operation 620 depicts presenting an indication of at least one artificial sensory experience and an indication of at least one inhalation therapy at least partially based on the accepting at least one indication of a health-related condition. For example, as shown in FIGS. 1 through 5, presenter module 104 may present an indication of a virtual world at least partially based on accepting an indication of a bioactive agent-dispensing inhalation device. One example of an artificial sensory experience may include a virtual world and/or other computer-simulated experience. Other examples of an artificial sensory experience may include experiences triggering sight, smell, hearing, touch, and/or taste. For example, presenter module 104 may present an indication of an artificial sensory experience including a virtual scent environment, which may include olfactory stimulation for improving memory. In an additional embodiment, presenter module 104 may present an indication of an artificial sensory experience including a virtual experience where the user is exposed to a virtual mountain environment coupled with a bronchoditator dose from a bioactive agent-dispensing inhalation collar. In this embodiment, the combination bronchodilator and virtual world treatment may serve to help an asthma sufferer to learn effective breathing techniques. Presenting an indication of an artificial sensory experience may include presenting the indication to a physician, to a computer monitor, to a mobile device, and/or to a third party. In some instances, presenter module 104 may include a computer processor and/or a communication device, such as a printer, a computer monitor, and/or a speaker.

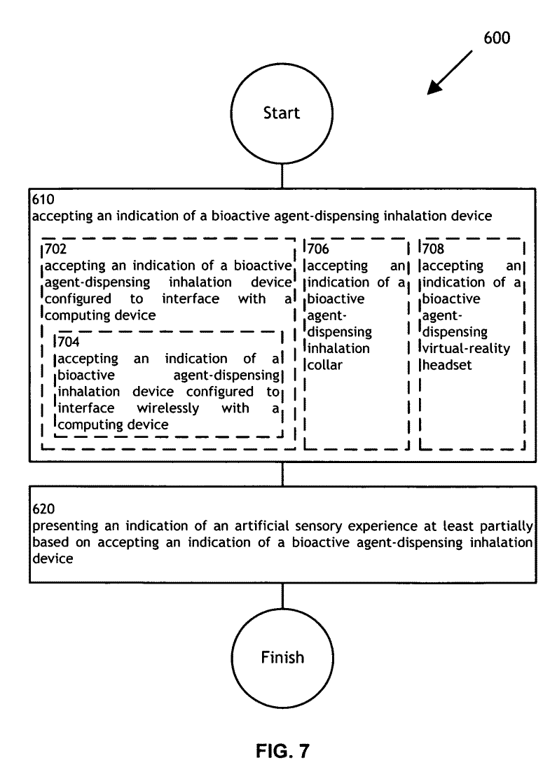

FIG. 7 illustrates alternative embodiments of the example operational flow 600 of FIG. 6. FIG. 7 illustrates example embodiments where operation 610 may include at least one additional operation. Additional operations may include operation 702, operation 704, operation 706, and/or operation 708.

Operation 702 illustrates accepting an indication of a health-related physical condition. For example, as shown in FIGS. 1 through 5, computer interfacing accepter module 402 may accept an indication of a bioactive agent-dispensing inhalation device configured to interface with a computing device. In one embodiment, computer interfacing accepter module 402 may accept an indication of a bioactive agent-dispensing inhalation device configured to interface with a virtual game, such as World of Warcraft. Some examples of a computing device may include a personal computer, a virtual-reality helmet and/or headset, and/or a virtual environment. In some instances, computer interfacing accepter module 402 may include a computer processor.

Further, operation 704 illustrates accepting an indication of a bioactive agent-dispensing inhalation device configured to interface wirelessly with a computing device. For example, as shown in FIGS. 1 through 5, wireless accepter module 404 may accept an indication of a bioactive agent-dispensing inhalation device configured to interface wirelessly with a computing device. In one embodiment, wireless accepter module 404 may accept an indication of a wireless inhalation collar configured to interface wirelessly with a computer coupled to wireless video glasses. In this embodiment, both the inhalation collar and the video glasses may be wirelessly connected to the computer. The wireless bioactive agent-dispensing inhalation device may be wirelessly coupled to a computing device using, for example, an IEEE 802.11 computer network and/or a Bluetooth wireless sensor network. One example of wireless video glasses may include Qingbar GP300 video glasses available from 22moo International Pty Ldt., Cabramatta NSW, Australia. In some instances, wireless accepter module 404 may include a computer processor and/or a wireless receiving device, such as a receiving antenna.

Operation 706 illustrates accepting an indication a health-related condition from a medical history. For example, as shown in FIGS. 1 through 5, inhalation collar indication accepter module 406 may accept an indication of a bioactive agent-dispensing inhalation collar. A bioactive agent-dispensing inhalation collar may include a collar with, for example, means for dispensing a bioactive agent, such as a reservoir and/or an accompanying valve and spray nozzle. Additionally, means for dispensing a bioactive agent may include means for dispensing an aerosol, vapor, a powder (e.g. pulmicort and/or foradil), and/or a mist, such as a nebulizer, means for measuring and/or detecting a condition, such as blood oxygen level and/or body temperature, and/or means for processing information, such as a computer processor and/or computer memory. Further, a bioactive agent may be dispensed and/or dispersed in and/or include a surfactant. In one embodiment, inhalation collar indication accepter module 406 may accept an indication of a bioactive agent-dispensing collar having means for dispensing a steroid as an aerosol. Further, a bioactive agent-dispensing inhalation collar may include means for power, such as a battery and/or circuitry for receiving power from an external source, such as an AC adapter power supply. In some instances, inhalation collar indication accepter module 406 may include a computer processor.

Operation 708 illustrates accepting an indication of a bioactive agent-dispensing virtual-reality headset. For example, as shown in FIGS. 1 through 5, headset indication accepter module 408 may accept an indication of a bioactive agent-dispensing virtual-reality headset. A virtual-reality headset may include a microphone, headphones or speakers for hearing, and/or a display. A virtual-reality headset may be configured for enabling a user to engage in an artificial sensory experience including sound, smell, and/or sight. One example of a virtual-reality headset may include a virtual reality helmet configured to give the user a 360° view of a mountain landscape while dispensing a bronchodilator for helping the user learn improved breathing techniques. Another example of a virtual reality head set may include an Olympus Eye-Trek FMD-200—TFT active matrix head mounted display with Speaker, available from Olympus America Inc., Center Valley Pa. In some instances, headset indication accepter module 408 may include a computer processor.

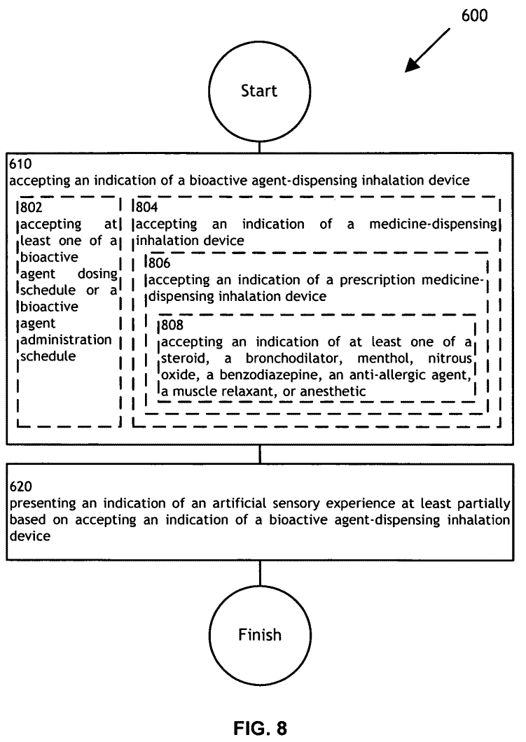

FIG. 8 illustrates alternative embodiments of the example operational flow 600 of FIG. 6. FIG. 8 illustrates example embodiments where the operation 610 may include at least one additional operation. Additional operations may include an operation 802, an operation 804, an operation 806, and/or an operation 808.

Operation 802 illustrates accepting an indication of a health-related mental condition. For example, as shown in FIGS. 1 through 5, schedule accepter module 410 may accept at least one of a bioactive agent dosing schedule or a bioactive agent administration schedule. Accepting a bioactive agent dosing schedule or a bioactive agent administration schedule may include accepting from a computer processor, accepting from a memory device, and/or accepting from a user input. In one embodiment, schedule accepter module 410 may accept a dosing schedule specifying a bronchodilator administration dosage for a specified time period, such one dose from an inhalation device once every thirty minutes. In another embodiment, schedule accepter module 410 may accept a bioactive agent administration schedule specifying at least one time a bronchodilator may be administered. In some instances, schedule accepter module 410 may include a computer processor.

Operation 804 illustrates accepting an indication of a medicine-dispensing inhalation device. For example, as shown in FIGS. 1 through 5, inhalation device accepter module 412 may accept an indication of a medicine-dispensing inhalation device. A medicine-dispensing inhalation device may include a device for dispensing a substance for treating a disease and/or illness. For example, a medicine-dispensing inhalation device may include an inhaler as described in Robertson et al., U.S. Pat. No. 7,383,837, which is incorporated herein by reference. Some other examples may include a metered-dose inhaler, a dry powder inhaler, and/or a nebulizer. In one embodiment, inhalation device accepter module 412 may accept an indication of a medicine-dispensing metered-dose inhaler configured to dispense albuterol. In some instances, inhalation device accepter module 412 may include a computer processor.

Further, operation 806 illustrates accepting an indication of a health-related condition from a user input. For example, as shown in FIGS. 1 through 5, prescription medicine device accepter module 414 may accept an indication of a prescription medicine-dispensing inhalation device. A prescription medicine-dispensing inhalation device may include a device configured to dispense a medication only available from a licensed health care provider. Some examples of a prescription medication available from a licensed health care provider may include albuterol, coricosteroids, nitrous oxide, a benzodiazepine, Theophylline, nedocromil sodium, and/or fluticasone/salmeterol. In one embodiment, prescription medicine device accepter module 414 may accept an indication of a prescription medicine-dispensing inhalation device configured for dispensing ciclesonide. In some instances, prescription medicine device accepter module 414 may include a computer processor.

Further, operation 808 illustrates indication of at least one of a prescribed artificial sensory experience or a prescribed inhalation therapy. For example, as shown in FIGS. 1 through 5, prescription medicine accepter module 416 may accept an indication of at least one of a steroid, a bronchodilator, menthol, nitrous oxide, a benzodiazepine, or halothane. One example of a steroid may include an anabolic steroid, which may be a derivative of androgens (such as testosterone), for stimulating growth. Another example of a steroid may include a corticosteroid, which may be often used as an anti-inflammatory prescribed for asthma. A bronchodilator may include a substance that dilates the bronchi and bronchioles decreasing airway resistance and thereby facilitating airflow. Menthol may include an organic and/or synthetic compound with local anesthetic and counterirritant qualities often used for relieving throat irritation and/or as a decongestant. Nitrous oxide may include a gas often used as a weak general anesthetic. A benzodiazepine may include a class of psychoactive drugs with varying hypnotic, sedative, anxiolytic, anticonvulsant, muscle relaxant and amnesic properties, which may be mediated by slowing down the central nervous system. In one embodiment, prescription medicine accepter module 416 may accept an indication of a benzodiazepine. One example of benzodiazepine delivery through an inhalation route may be disclosed in Kim et al., U.S. Patent Publication No. 2003/0032638, which is incorporated herein by reference. An anti-allergic agent may include an agent configured to block the action of allergic mediators and/or to prevent activation of cells and degranulation processes. Some examples of an anti-allergic agent may include an antihistamine and/or cromones like mast cell stabilizers, such as cromoglicic acid and nedocromil sodium. A muscle relaxant may include a bioactive agent for affecting skeletal muscle function and/or decreasing muscle tone. One example of a skeletal muscle relaxant may include carisoprodol. Additionally, a muscle relaxant may include a smooth muscle relaxant. One example of a smooth muscle relaxant may include a methylxanthine, such as Theophylline. An anesthetic may include an inhalational general anesthetic, such as halothane, desflurane, enflurane, isoflurane, and/or sevoflurane. In some instances, prescription medicine accepter module 416 may include a computer processor.

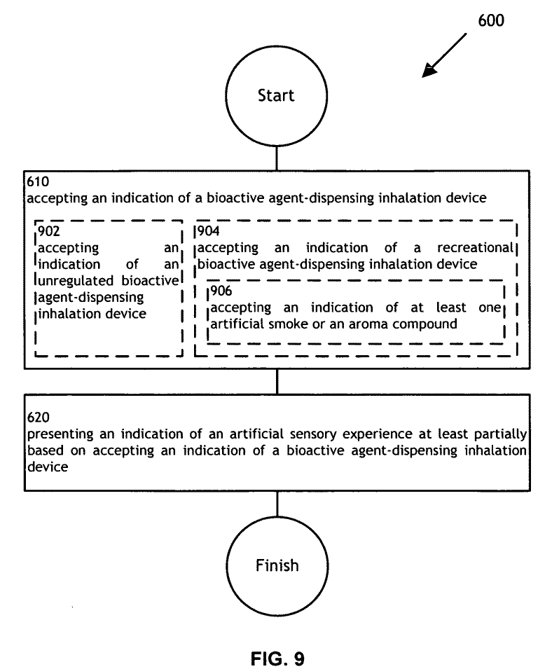

FIG. 9 illustrates alternative embodiments of the example operational flow 600 of FIG. 6. FIG. 9 illustrates example embodiments where the operation 610 may include at least one additional operation. Additional operations may include an operation 902, an operation 904, and/or an operation 906.

Operation 902 illustrates accepting an indication of an unregulated bioactive agent-dispensing inhalation device. For example, as shown in FIGS. 1 through 5, unregulated device accepter module 418 may accept an indication of an unregulated bioactive agent-dispensing inhalation device. In one embodiment, unregulated device accepter module 418 may accept an indication of an oxygen-dispensing inhalation device. Some examples of an unregulated bioactive agent may include oxygen, aromas used for aromatherapy, and/or menthol. In another embodiment, unregulated device accepter module 418 may accept an indication of an aromatherapeutic-dispensing inhalation collar. In some instances, unregulated device accepter module 418 may include a computer processor.

Operation 904 illustrates accepting an indication of a recreational bioactive agent-dispensing inhalation device. For example, as shown in FIGS. 1 through 5, recreational device accepter module 420 may accept an indication of a recreational bioactive agent-dispensing inhalation device. In one embodiment, recreational device accepter module 420 may accept an indication of a recreational bioactive agent-dispensing inhalation device. Some examples of a recreational bioactive agent may include an aroma compound used for aromatherapy and/or artificial smoke. Other examples of a recreational bioactive agent may include incense and/or smoke, such as incense and/or smoke used in a religious rite. In some instances, recreational device accepter module 420 may include a computer processor.

Further, operation 906 illustrates accepting an indication of at least one artificial smoke or an aroma compound. For example, as shown in FIGS. 1 through 5, recreational compound indication accepter module 422 may accept an indication of at least one artificial smoke or an aroma compound. In one embodiment, recreational compound indication accepter module 422 may accept an indication of artificial smoke while experiencing a virtual world. In another embodiment, recreational compound indication accepter module 422 may accept an indication of lemon oil while experiencing an artificial sensory experience. In this embodiment, the use of lemon oil as an aromatherapeutic may serve to enhance a user's mood and/or provide relaxation. In some instances, recreational compound indication accepter module 422 may include a computer processor.

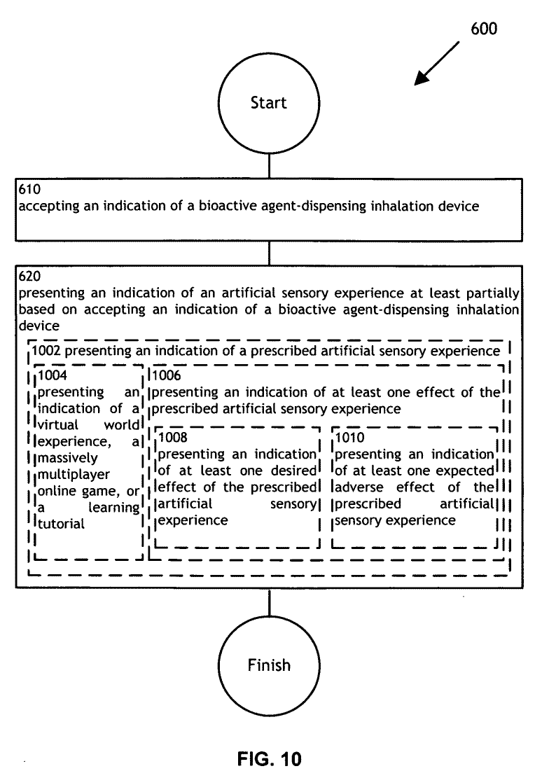

FIG. 10 illustrates alternative embodiments of the example operational flow 600 of FIG. 6. FIG. 10 illustrates example embodiments where operation 620 may include at least one additional operation. Additional operations may include operation 1002, operation 1004, operation 1006, operation 1008, and/or operation 1010.

Operation 1002 illustrates indication of at least one of a prescribed artificial sensory experience or a prescribed inhalation therapy. For example, as shown in FIGS. 1 through 5, prescription artificial sensory experience presenter module 424 may present an indication of a prescribed artificial sensory experience. A prescribed artificial sensory experience may include any artificial sensory experience prescribed by a health care professional, such as a physician, a mental health specialist, a nurse, a physical therapist, an occupational therapist, a chiropractor, and/or a homeopathic practitioner. In one embodiment, prescription artificial sensory experience presenter module 424 may present an indication of a virtual world prescribed by a psychiatrist. In this embodiment, the prescribed virtual world may be configured to be administered in conjunction with a prescribed bioactive agent. Administering a prescribed bioactive agent in conjunction with a prescribed artificial sensory experience may serve to increase efficacy of the combined therapy, for example, by serving as a distraction from pain. In some instances, prescription artificial sensory experience presenter module 424 may include a computer processor and/or a display device, such as a computer monitor and/or a printer.

Further, operation 1004 illustrates an indication of at least one of a virtual world experience, a massively multiplayer online game, or a learning tutorial. For example, as shown in FIGS. 1 through 5, artificial sensory experience presenter module 426 may present an indication of a virtual world experience, a massively multiplayer online game, or a learning tutorial. A virtual world experience may include a computer-based simulated environment intended to be interactive. Some examples of a virtual world experience may include a text-based chat room, computer conferencing, an online game, a single player game, and/or a computer tutorial. A massively multiplayer online game may include a video game capable of supporting multiple players, such as World of Warcraft and/or SecondLife. Additionally, a massively multiplayer online game may include an experience, such as a game, which may include a video game or other interactive experience involving numbers of individuals, for example, a religious ceremony or combat training exercise. An online learning tutorial may include a screen recording, a written document (either online or downloadable), or an audio file, where a user may be given step by step instructions on how to do something. In one embodiment, artificial sensory experience presenter module 426 may present an indication of a virtual world experience, such as World of Warcraft. In some instances, artificial sensory experience presenter module 426 may include a computer processor.

Further, operation 1006 illustrates indication of at least one effect of the indication of at least one of a prescribed artificial sensory experience. For example, as shown in FIGS. 1 through 5, artificial sensory experience effect presenter module 428 may present an indication of at least one effect of the prescribed artificial sensory experience. In one embodiment, artificial sensory experience effect presenter module 428 may present an indication of at least one effect of the prescribed artificial sensory experience. An effect may include a reaction and/or thing that occurs as a result of the artificial sensory experience. For example, an effect may include a side effect, a desired effect, and/or an adverse effect. Some examples of an effect may include an increased bioactive agent efficacy, dizziness, and/or a decreased heart rate. In some instances, artificial sensory experience effect presenter module 428 may include a computer processor.

Further, operation 1008 illustrates presenting an indication of at least one expected desired effect of the prescribed artificial sensory experience. For example, as shown in FIGS. 1 through 5, artificial sensory experience desired effect presenter module 430 may present an indication of at least one desired effect of the prescribed artificial sensory experience. Some examples of a desired effect may include effects such as an increased bioactive agent efficacy, a cured illness and/or condition, and/or a changed behavior. In one embodiment, artificial sensory experience desired effect presenter module 430 may present an indication of an increased opioid efficacy measured by self pain evaluation by an individual. In some instances, artificial sensory experience desired effect presenter module 430 may include a computer processor and/or a display, such as a monitor and/or a printer.

Further, operation 1010 illustrates an indication of at least one prescribed inhalation therapy. For example, as shown in FIGS. 1 through 5, artificial sensory experience adverse effect presenter module 432 may present an indication of an expected adverse effect of the prescribed artificial sensory experience. An adverse effect may include a harmful and/or undesired effect resulting from an intervention, such as an artificial sensory experience. Some examples of an adverse effect may include headache, dizziness, depression, bleeding, seizure, and/or fever. In one embodiment, artificial sensory experience adverse effect presenter module 432 may present an indication of fever in an individual while being administered a prescribed artificial sensory experience and bioactive agent. In some instances, artificial sensory experience adverse effect presenter module 432 may include a computer processor, a display device, such as a monitor and/or printer, and/or medical instrumentation, such as a thermometer configured for measuring a body temperature.

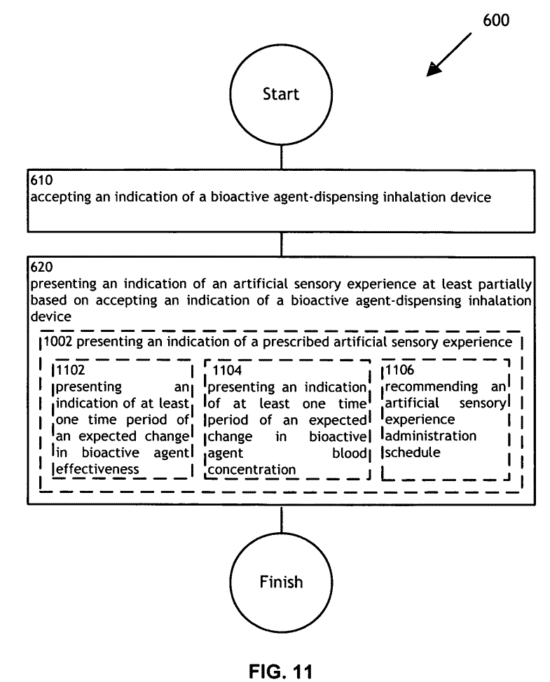

FIG. 11 illustrates alternative embodiments of the example operational flow 600 of FIG. 6. FIG. 11 illustrates example embodiments where operation 620 may include at least one additional operation. Additional operations may include operation 1102, operation 1104, and/or operation 1106.

Operation 1102 illustrates an indication of at least one prescribed bioactive agent. For example, as shown in FIGS. 1 through 5, effectiveness change presenter module 434 may present an indication of at least one time period of an expected change in bioactive agent effectiveness. In one embodiment, effectiveness change presenter module 434 may present an indication of a time period when an opioid is expected to decrease in effectiveness. Such an indication of decrease and/or change in bioactive agent effectiveness may serve to indicate an appropriate time period for administering and/or modifying an artificial sensory experience to compensate for a change in bioactive agent efficacy. In another embodiment, effectiveness change presenter module 434 may present an indication of a time period where a blood stream morphine concentration drops. This time period of low blood stream morphine concentration may be appropriate for presenting an immersive virtual world for serving as a distraction to any increase in pain caused by lowered morphine concentration. In some instances, effectiveness change presenter module 434 may include a computer processor.

Further, operation 1104 illustrates an indication of at least one time period of an expected change in bioactive agent blood concentration. For example, as shown in FIGS. 1 through 5, concentration change presenter module 436 may present an indication of at least one time period of an expected change in bioactive agent blood concentration. In one embodiment, concentration change presenter module 436 may present an indication of a one hour time period of an expected change in hydrocodone blood concentration. Indicating a time period of a change in blood concentration may serve to help determine an artificial sensory experience administration schedule. For example, if a bioactive agent blood concentration is expected to be reduced during a certain time period, an artificial sensory experience configured for distracting an individual from pain may be selected for administration during that time period. In some instances, concentration change presenter module 436 may include a computer processor and/or a display device, such as a printer and/or a computer monitor.

Further, operation 1106 illustrates recommending at least one of an artificial sensory experience administration schedule. For example, as shown in FIGS. 1 through 5, recommender module 438 may recommend an artificial sensory experience administration schedule. In one embodiment, recommender module 438 may recommend a time schedule for administration of a virtual world experience. A time schedule may be recommended by taking into account factors involving the individual and/or the bioactive agent. For example, efficacy of the bioactive agent versus time may be a factor, such as a time period when the bioactive agent is less effective. Efficacy of the bioactive agent may be a factor in determining when an artificial sensory experience is administered because of the potential for the artificial sensory experience to compensate for a changed bioactive agent efficacy. An additional factor may include an attribute of the individual, such as how a bioactive agent and/or specific artificial sensory experience affects the individual, for example a side effect. Another example of recommending an artificial sensory experience may be found in Akazawa et al., U.S. Pat. No. 7,155,680, which is incorporated herein by reference. In some instances, recommender module 438 may include a computer processor.

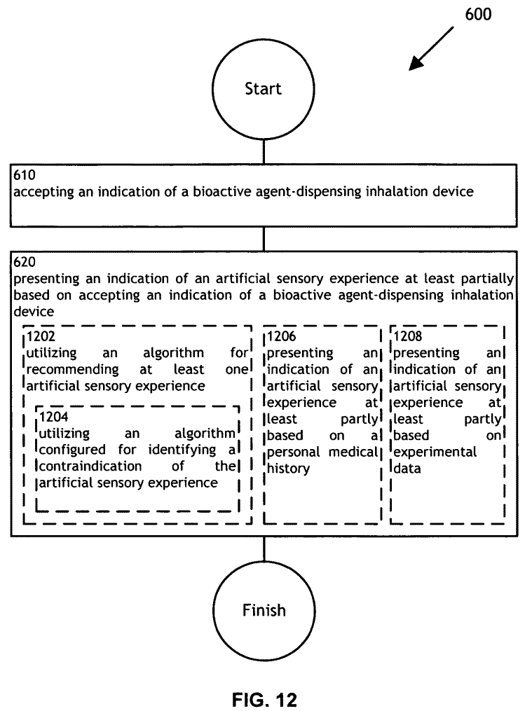

FIG. 12 illustrates alternative embodiments of the example operational flow 600 of FIG. 6. FIG. 12 illustrates example embodiments where operation 620 may include at least one additional operation. Additional operations may include operation 1202, operation 1204, operation 1206, and/or operation 1208.

Operation 1202 illustrates an indication of an unregulated inhalation. For example, as shown in FIGS. 1 through 5, algorithm utilizer module 440 may utilize an algorithm for recommending at least one artificial sensory experience. An algorithm for recommending an artificial sensory experience may include any computation, formula, statistical survey, and/or look-up table for determining and/or selecting a suitable artificial sensory experience. Some examples may include a computer software algorithm, a calculator, a flowchart, and/or a decision tree. In one embodiment, algorithm utilizer module 440 may utilize an algorithm that uses an inputted indication of an analgesic, such as oxycodone, and determines a suitable artificial sensory experience by analyzing periods of low blood concentration of the oxycodone. In this embodiment, algorithm utilizer module 440 may recommend an artificial sensory experience that may be effective in pain distraction when bioactive agent blood concentration may be reduced but before an additional dose may be available. In some instances, algorithm utilizer module 440 may include a computer processor.

Further, operation 1204 illustrates an indication of an unregulated inhalation. For example, as shown in FIGS. 1 through 5, contraindication algorithm utilizer module 442 may utilize an algorithm configured for identifying a contraindication of the artificial sensory experience. A contraindication of an artificial sensory experience may include giving an indication against the advisability of the artificial sensory experience. For example, contraindication algorithm utilizer module 442 may utilize an algorithm that considers an individual's personal medical history, such as a phobia, and may recommend not prescribing a certain artificial sensory experience, which may include an object that may trigger the phobia. Contraindication algorithm utilizer module 442 may identify a contraindication of an artificial sensory experience for reasons such as an adverse effect and/or inefficacy. In some instances, contraindication algorithm utilizer module 442 may include a computer processor.

Operation 1206 illustrates presenting an indication of an artificial sensory experience at least partly based on a personal medical history. For example, as shown in FIGS. 1 through 5, medical history indication presenter module 444 may present an indication of an artificial sensory experience at least partly based on a personal medical history. A medical history may include a personal history and/or a family history. A personal medical history may include a list of previous illnesses, symptoms, medicines, treatments, health risk factors, operations, and/or doctor visits associated with at least one individual. A personal and/or a family medical history may include life history and/or social history characteristics such as smoking, drinking, drug use, sexual history, exercise history, eating history, nutraceutical history, or the like. In one embodiment, medical history indication presenter module 444 may present an indication of a suitable virtual world based on a personal medical history. In this embodiment, the personal medical history may indicate that an individual may be averse to a certain virtual world, such as a virtual world with rapid animation that may cause nausea. In some instances, medical history indication presenter module 444 may include a computer processor and/or a display device, such as a computer monitor and/or a printer.

Operation 1208 illustrates utilizing an algorithm configured for recommending at least one of an artificial sensory experience. For example, as shown in FIGS. 1 through 5, experimental data indication presenter module 446 may present an indication of an artificial sensory experience at least partly based on experimental data. Experimental data may include any data from an experiment, such as a clinical trial. The experiment may be an experiment including an individual and/or a group of people. In one embodiment, experimental data indication presenter module 446 may present an indication of a virtual world suitable for an individual based on a clinical trial involving a group of 1,000 people showing a certain success rate for reducing a phobia, such as fear of heights. In some instances, experimental data indication presenter module 446 may include a computer processor and/or a display device, such as a computer monitor, a mobile phone, and/or a printer.

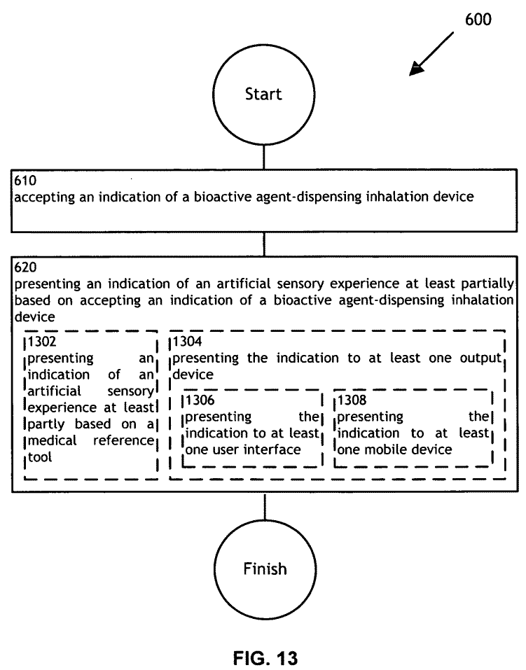

FIG. 13 illustrates alternative embodiments of the example operational flow 600 of FIG. 6. FIG. 13 illustrates example embodiments where the operation 620 may include at least one additional operation. Additional operations may include an operation 1302, an operation 1304, an operation 1306, and/or an operation 1308.

Operation 1302 illustrates presenting at least one of an indication of an artificial sensory experience or an indication of inhalation therapy at least partly based on a medical reference tool. For example, as shown in FIGS. 1 through 5, reference tool indication presenter module 448 may present an indication of an artificial sensory experience at least partly based on a medical reference tool. A medical reference tool may include a reference book, a reference database, and/or reference software. Some examples of a medical reference book may include a medical dictionary, a medical journal, and/or a book of drug interactions. One example of a reference database may include the National Cancer Center Cancer Image Reference (NCC-CIR) database and/or DynaMed. Some examples of reference software may include Skyscape software for a mobile phone and/or MedALert. In one embodiment, reference tool indication presenter module 448 may present an indication of an artificial sensory experience based on a reference database, such as a database including data from a clinical trial. In some instances, reference tool indication presenter module 448 may include a computer processor and/or a display device, such as a mobile phone, a printer, and/or a computer monitor.

Operation 1304 illustrates presenting the indication to at least one output device. For example, as shown in FIGS. 1 through 5, output device presenter module 450 may present to at least one output device. In one example, output device presenter module 450 may present an indication of a combination prescription medication and an artificial sensory experience therapy to an output device 130, such as a printer and/or monitor at a health clinic. An output device may include any hardware device configured for receiving computer output. Some examples of an output device may include a printer, a monitor, a mobile phone, a speaker, and/or a visual display unit. The output device 130 may be used by individual 134. In some instances, output device presenter module 450 may include a computer processor.

Further, operation 1306 illustrates presenting the indication to at least one user interface. For example, as shown in FIGS. 1 through 5, user interface presenter module 452 may present to at least one user interface. In one embodiment, user interface presenter module 452 may present to a touchscreen device. A user interface may include means by which an individual may interact with a system. Some examples of a user interface may include a touchscreen, a graphical user interface, a tactile interface, and/or a live user interface. In some instances, user interface presenter module 452 may include a computer processor.

Further, operation 1308 illustrates presenting the indication to at least one mobile device. For example, as shown in FIGS. 1 through 5, mobile device presenter module 454 may present to at least one mobile device. In one embodiment, mobile device presenter module 454 may present to a mobile phone. A mobile device may include a portable computing device and may have wireless connection capability. Some examples of a mobile device may include a laptop or notebook computer, a personal digital assistant (PDA), an ipod, a smartphone, an Enterprise digital assistant (EDA), and/or a pager. In some instances, mobile device presenter module 454 may include a computer processor.

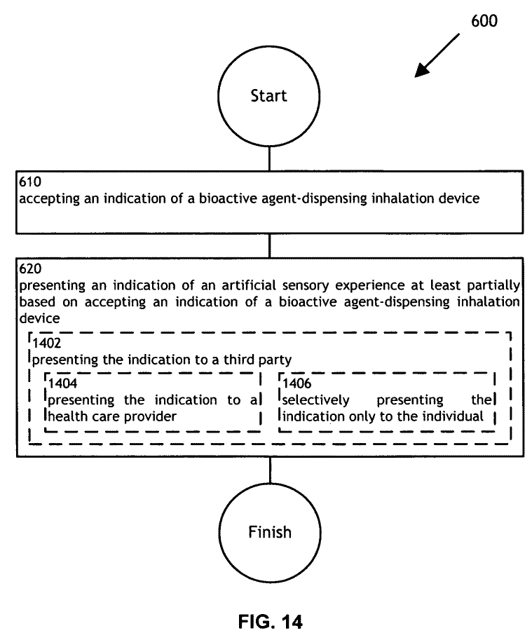

FIG. 14 illustrates alternative embodiments of the example operational flow 600 of FIG. 6. FIG. 14 illustrates example embodiments where operation 620 may include at least one additional operation. Additional operations may include operation 1402, operation 1404, and/or operation 1406.

Operation 1402 illustrates presenting the indication to at least one third party. For example, as shown in FIGS. 1 through 5, third party presenter module 456 may present to an individual's physician. A third party may include a party that is an independent party, person, and/or entity. Some examples of a third party may include a physician, a medical database, a hospital, a law enforcement agency, and/or a pharmacy. In one embodiment, third party presenter module 456 may present an indication to an insurance company. Another example of reporting to a third party may include creating displays and reports for aggregating data from therapy results, further discussed in Bair et al., U.S. Pat. No. 6,067,523, which is incorporated herein by reference. In some instances, third party presenter module 456 may include a computer processor and/or a communications device, such as a monitor and network link.

Further, operation 1404 illustrates presenting the indication to at least one health care provider. For example, as shown in FIGS. 1 through 5, health care provider presenter module 458 may present to a health care provider. A health care provider may include a pharmacy, a pharmaceutical company, a medical device company, a research institution, a computer software and/or computer hardware company, a website, a nurse and/or a physician. In one embodiment, health care provider presenter module 458 may present to a physician a prescribed combination artificial sensory experience and bioactive agent therapy via a secured website. In some instances, health care provider presenter module 458 may include a computer processor.

Further, operation 1406 illustrates selectively presenting the indication only to the individual. For example, as shown in FIGS. 1 through 5, selective presenter module 460 may selectively present only to the individual. Selective presenting may include limiting and/or blocking access of an individual's compliance results and/or a prescribed therapy, such as a prescribed artificial sensory experience and/or bioactive agent to a specific party. For example, selective presenter module 460 may present only to individual 134 and may keep results of a certain combination therapy confidential. In one embodiment, an encryption key may be employed to protect selected information. In an additional example, selective presenter module 460 may report only to a law enforcement agency and/or representative, such as a probation officer, and not to individual 134. In some instances, selective presenter module 460 may include a computer processor.

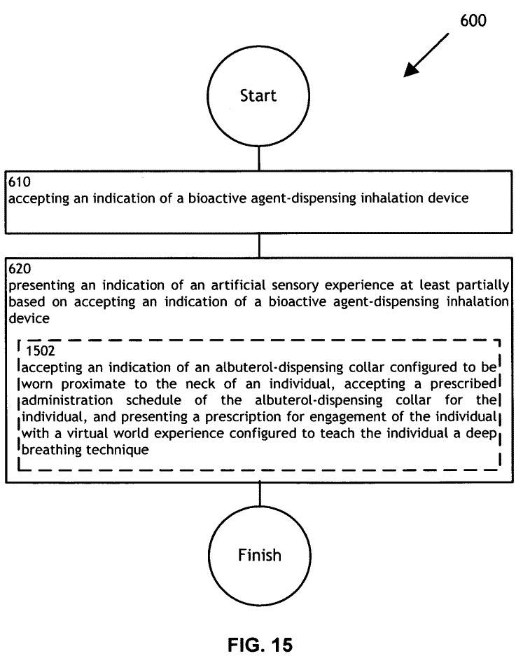

FIG. 15 illustrates alternative embodiments of the example operational flow 600 of FIG. 6. FIG. 15 illustrates example embodiments where the operation 620 may include at least one additional operation. Additional operations may include an operation 1502.

Operation 1502 illustrates accepting an indication of an individual's asthma, presenting a prescribed administration schedule of an albuterol-dispensing collar therapy for the individual, and presenting a prescription for engagement of the individual with a virtual world experience configured to teach the individual a deep breathing technique. For example, as shown in FIGS. 1 through 5, accepter module 102 and/or presenter module 104 may accept an indication of an albuterol-dispensing collar configured to be worn proximate to the neck of an individual, accept a prescribed administration schedule of the albuterol-dispensing collar for the individual, and present a prescription for engagement of the individual with a virtual world experience configured to teach the individual a deep breathing technique. In some instances, accepter module 102 and/or presenter module 104 may include a computer processor.

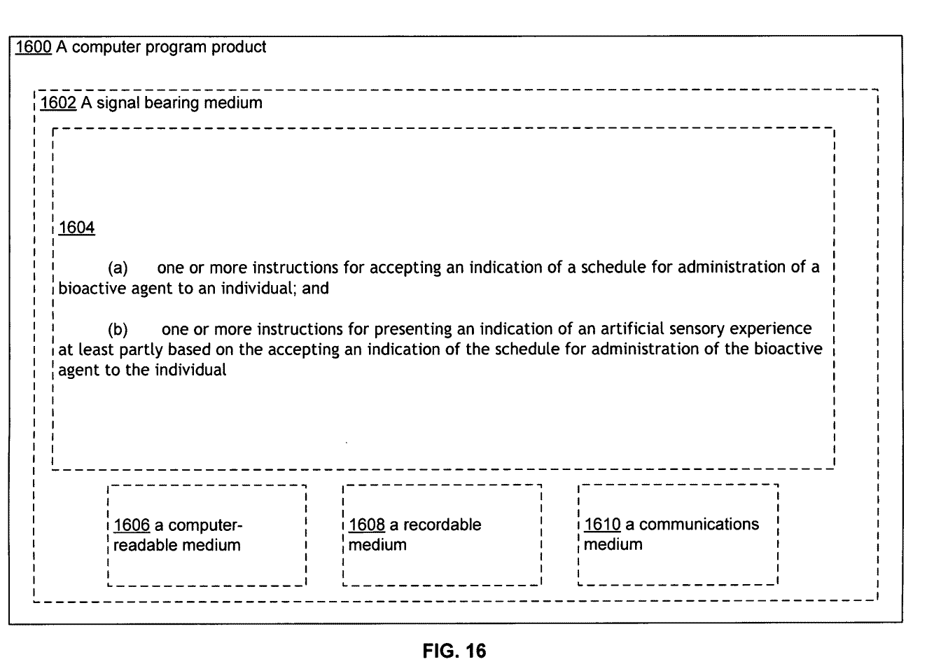

FIG. 16 illustrates a partial view of an example computer program product 1600 that includes a computer program 1604 for executing a computer process on a computing device. An embodiment of the example computer program product 1600 is provided using a signal-bearing medium bearing 1602, and may include one or more instructions for accepting an indication of at least one health-related condition and one or more instructions for presenting an indication of at least one artificial sensory experience and an indication of at least one inhalation therapy at least partially based on the accepting at least one indication of a health-related condition. The one or more instructions may be, for example, computer executable and/or logic-implemented instructions. In one implementation, the signal-bearing medium 1602 may include a computer-readable medium 1606. In one implementation, the signal bearing medium 1602 may include a recordable medium 1608. In one implementation, the signal bearing medium 1602 may include a communications medium 1610.

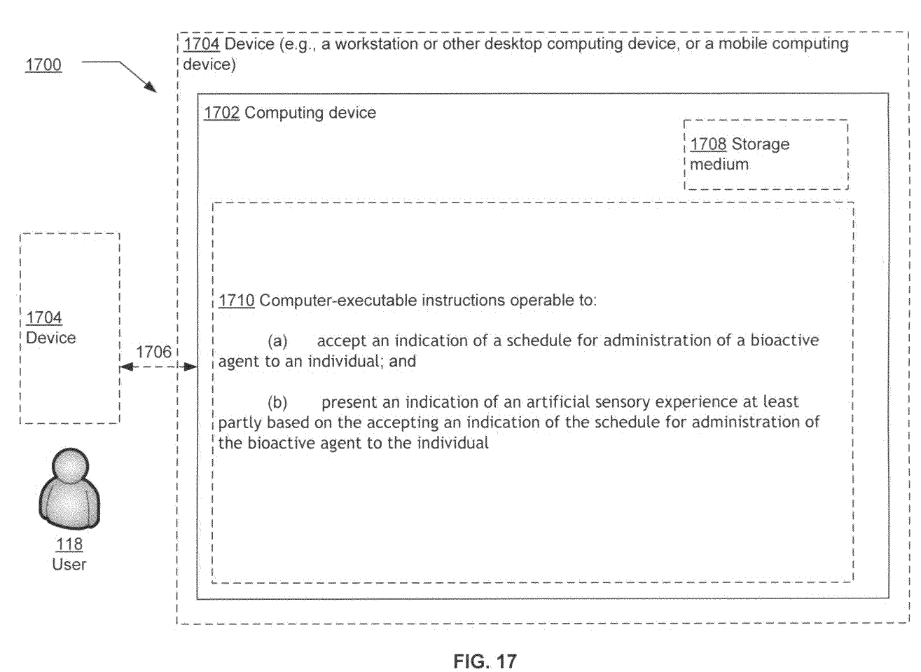

FIG. 17 illustrates an example system 1700 in which embodiments may be implemented. The system 1700 includes a computing system environment. The system 1700 also illustrates the user 118 using a device 1704, which is optionally shown as being in communication with a computing device 1702 by way of an optional coupling 1706. The optional coupling 1706 may represent a local, wide-area, or peer-to-peer network, or may represent a bus that is internal to a computing device (e.g., in example embodiments in which the computing device 1702 is contained in whole or in part within the device 1704). A storage medium 1708 may be any computer storage media.

The computing device 1702 includes computer-executable instructions 1710 that when executed on the computing device 1702 cause the computing device 1702 to accept an indication of a schedule for administration of a bioactive agent to an individual and present an indication of an artificial sensory experience at least partly based on the accepting an indication of the schedule for administration of the bioactive agent to the individual. As referenced above and as shown in FIG. 17, in some examples, the computing device 1702 may optionally be contained in whole or in part within the device 1704.

In FIG. 17, then, the system 1700 includes at least one computing device (e.g., 1702 and/or 1704). The computer-executable instructions 1710 may be executed on one or more of the at least one computing device. For example, the computing device 1702 may implement the computer-executable instructions 1710 and output a result to (and/or receive data from) the computing device 1704. Since the computing device 1702 may be wholly or partially contained within the computing device 1704, the device 1704 also may be said to execute some or all of the computer-executable instructions 1710, in order to be caused to perform or implement, for example, various ones of the techniques described herein, or other techniques.

The device 1704 may include, for example, a portable computing device, workstation, or desktop computing device. In another example embodiment, the computing device 1702 is operable to communicate with the device 1704 associated with the user 118 to receive information about the input from the user 118 for performing data access and data processing and presenting an output of the user-health test function at least partly based on the user data.

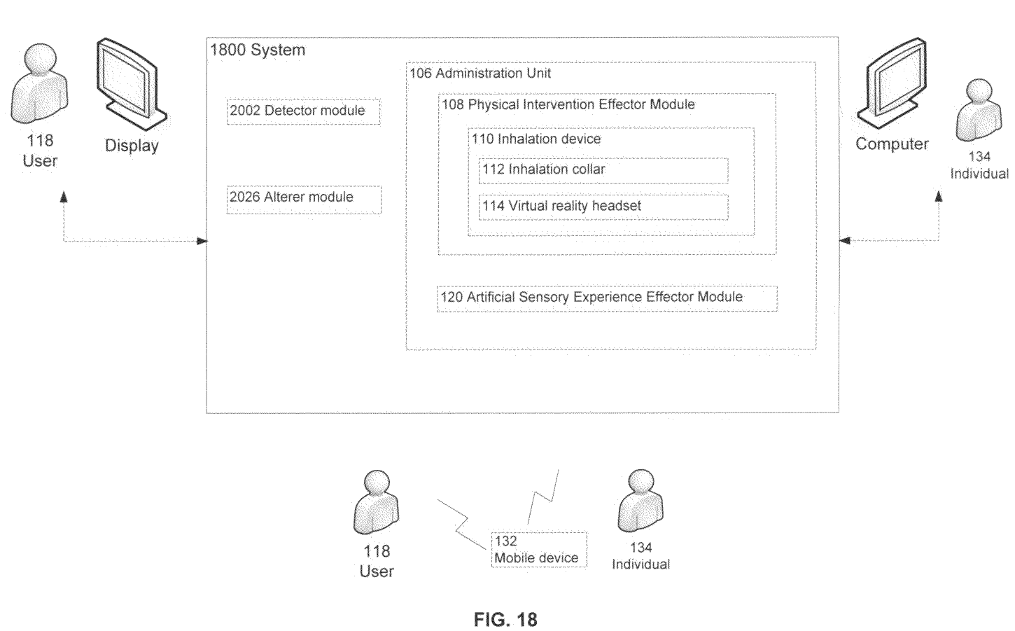

FIG. 18 illustrates system 1800 for detecting in an exhalant at least one indication of bioactive agent use by an individual and/or altering at least one artificial sensory experience at least partly based on the at least one indication of bioactive agent use by an individual. System 1800 may include detector module 2002, alterer module 2026, and/or administration unit 106. Administration unit 106 may include physical intervention effector module 108 and/or artificial sensory experience effector module 120. Physical intervention effector module 108 may include inhalation device 110. Inhalation device 110 may include inhalation collar 112 and/or virtual reality headset 114. Additionally, system 1800 may include mobile device 132.

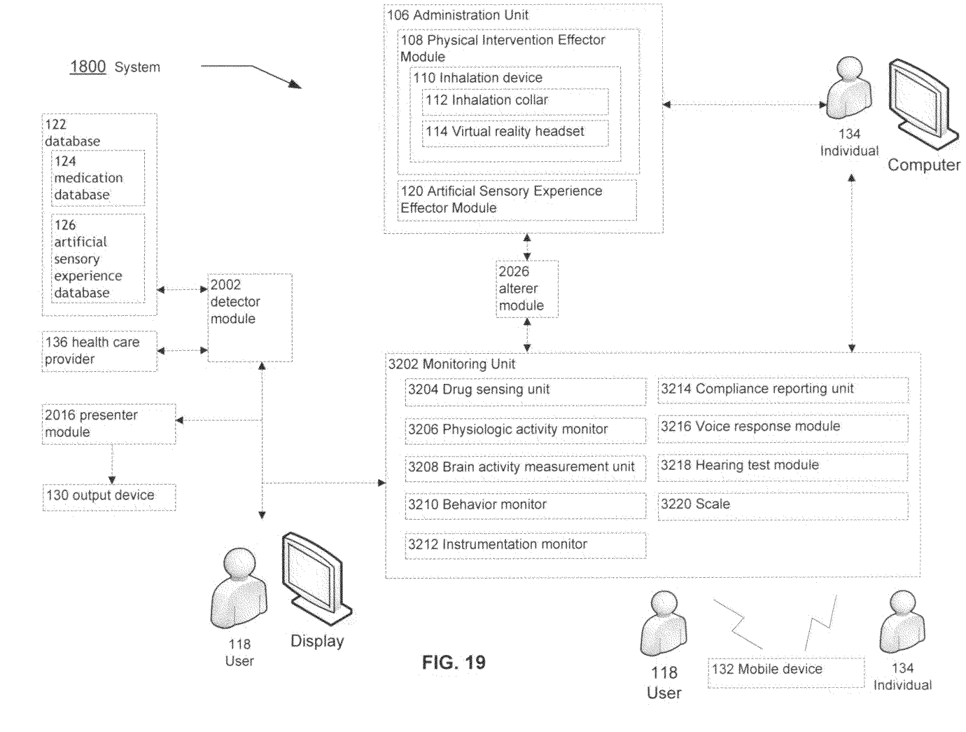

FIG. 19 illustrates system 1800 for detecting in an exhalant at least one indication of bioactive agent use by an individual and/or altering at least one artificial sensory experience at least partly based on the at least one indication of bioactive agent use by an individual. System 1800 may include detector module 2002, alterer module 2026, administration unit 106, and/or monitoring unit 3202. Detector module 2002 may receive and/or transmit information and/or data to and/or from user 118, database 122, presenter module 2016, output device 130, and/or health care provider 136. A user may include individual 134, health care provider 136, a patient, and/or another affected person and/or entity. Database 122 may include medication database 124 and/or artificial sensory experience database 126. Monitoring unit 3202 may monitor individual 134 and may include drug sensing unit 3204, physiologic activity monitor 3206, brain activity measurement unit 3208, behavior monitor 3210, instrumentation monitor 3212, compliance reporting unit 3214, voice response module 3216, hearing test module 3218, and/or scale 3220. Administration unit 106 may include physical intervention effector module 108 and/or artificial sensory experience effector module 120. Physical intervention effector module 108 may include inhalation device 110. Inhalation device 110 may include inhalation collar 112 and/or virtual reality headset 114. Additionally, mobile device 132 may communicate with detector module 2002, presenter module 2016, healthcare provider 136, user 118, individual 134, monitoring unit 3202, alterer module 2026, and/or administration unit 3222.

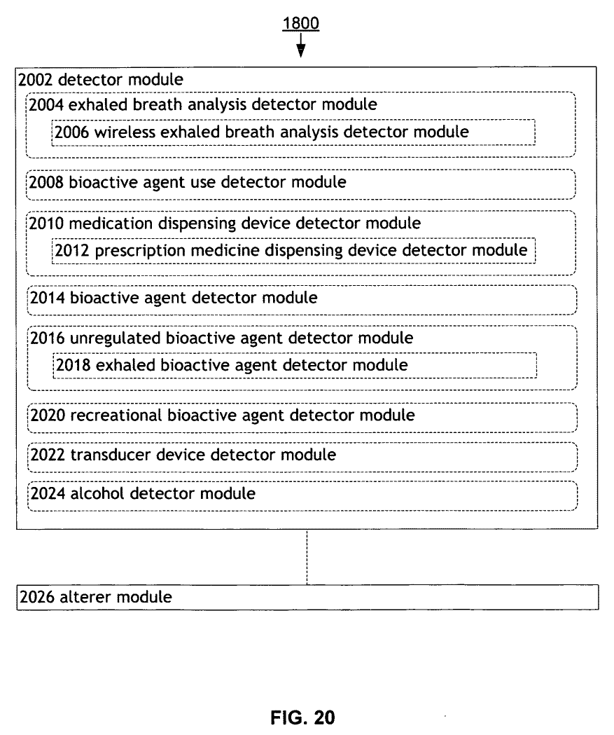

FIG. 20 further illustrates system 1800 including detector module 2002 and/or alterer module 2026. Detector module 2002 may include exhaled breath analysis detector module 2004, bioactive agent use detector module 2008, medication dispensing device detector module 2010, bioactive agent detector module 2014, unregulated bioactive agent detector module 2016, recreational bioactive agent detector module 2020, transducer device detector module 2022, and/or alcohol detector module 2024. Exhaled breath analysis detector module 2004 may include wireless exhaled breath analysis detector module 2006. Medicine dispensing device detector module 2010 may include prescription medicine dispensing device detector module 2012. Unregulated bioactive agent detector module 2016 may include exhaled bioactive agent detector module 2018.

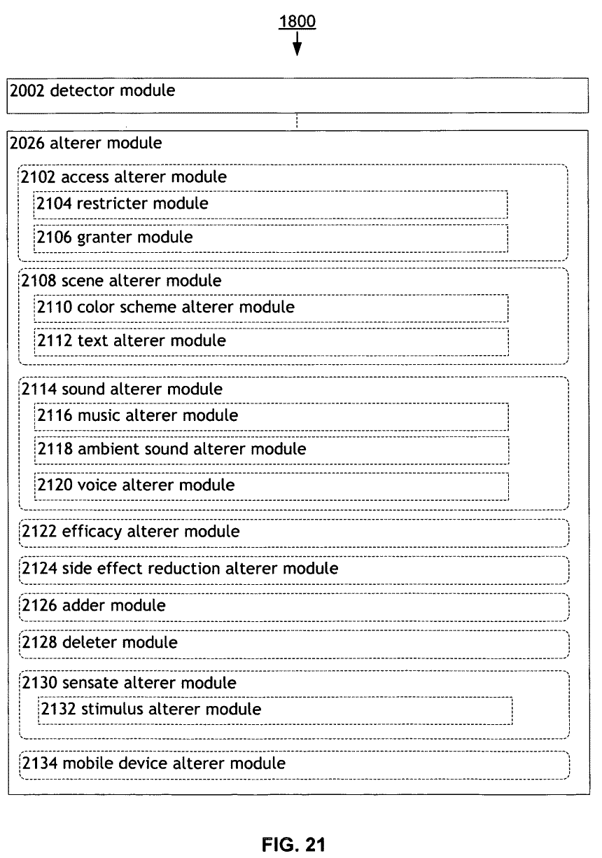

FIG. 21 further illustrates system 1800 including detector module 2002 and/or alterer module 2026. Alterer module 2026 may include access alterer module 2102, visual object alterer module 2108, sound alterer module 2114, efficacy alterer module 2122, side effect reduction alterer module 2124, adder module 2126, deleter module 2128, sensate alterer module 2130, and/or mobile device alterer module 2134. Access alterer module 2102 may include restricter module 2104 and/or granter module 2106. Visual object alterer module 2108 may include color scheme alterer module 2110 and/or text alterer module 2112. Sound alterer module 2114 may include music alterer module 2116, ambient noise alterer module 2118, and/or voice alterer module 2120. Sensate alterer module 2130 may include stimulus alterer module 2132.

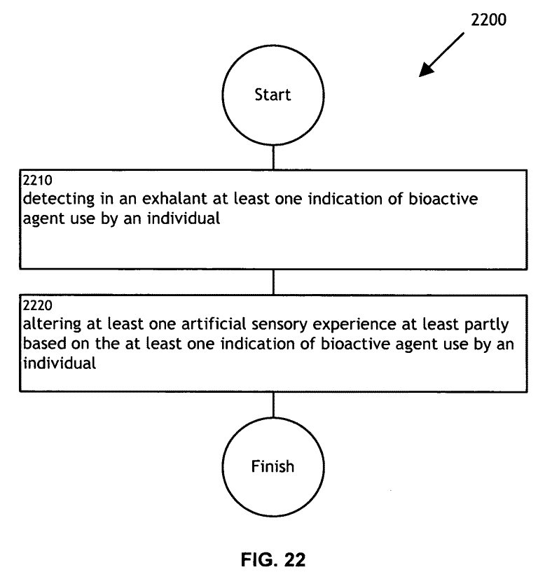

FIG. 22 illustrates operational flow 2200 representing example operations related to detecting in an exhalant at least one indication of bioactive agent use by an individual and altering at least one artificial sensory experience at least partly based on the at least one indication of bioactive agent use by an individual. In FIG. 22 and in following figures that include various examples of operational flows, discussion and explanation may be provided with respect to the above-described examples of FIGS. 18 through 21, and/or with respect to other examples and contexts. However, it should be understood that the operational flows may be executed in a number of other environments and contexts, and/or in modified versions of FIGS. 18 through 21. Also, although the various operational flows are presented in the sequence(s) illustrated, it should be understood that the various operations may be performed in other orders than those which are illustrated, or may be performed concurrently.

After a start operation, operational flow 2200 moves to operation 2210. Operation 2210 depicts detecting in an exhalant at least one indication of bioactive agent use by an individual. For example, as shown in FIGS. 18 through 21, detector module 2002 may detect in an exhalant at least one indication of bioactive agent use by an individual. In one embodiment, detector module 2002 may detect in an exhaled breath an indication of prescription steroid use. In this embodiment, detecting an indication of prescription steroid use and/or other bioactive agent use in an individual may serve to provide more detailed information for tailoring the prescription steroid use to an individual's needs. In some instances, detector module 2002 may include a computer processor and/or other detecting instrumentation (e.g., a chemical analysis kit, a transducer, and/or a gas analyzer).

Then, operation 2220 depicts altering at least one artificial sensory experience at least partly based on the at least one indication of bioactive agent use by an individual. For example, as shown in FIGS. 18 through 21, alterer module 2026 may alter at least one artificial sensory experience at least partly based on the at least one indication of bioactive agent use by an individual. In one embodiment, alterer module 2026 may alter music in a virtual world to create a more relaxing environment for a patient/participant based on an indication of a prescribed anti-anxiety medication administered to the individual. Altering an artificial sensory experience based on an indication of bioactive agent use by an individual may serve to modify an effect and/or efficacy of the bioactive agent. For example, the above altered music may create a more relaxing environment for an individual, which may amplify the effects of the anti-anxiety medication. In some instances, alterer module 2026 may include a computer processor and/or alteration instrumentation, such as a light dimmer switch, a speaker system, and/or a monitor.

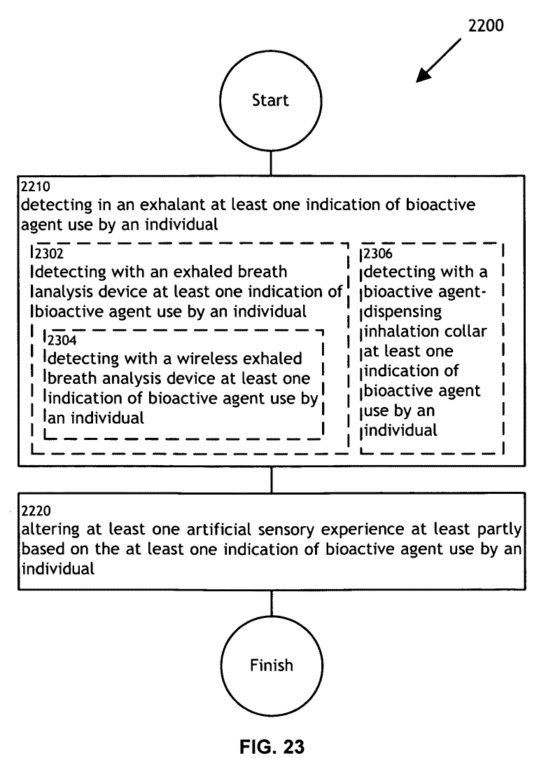

FIG. 23 illustrates alternative embodiments of the example operational flow 2200 of FIG. 22. FIG. 23 illustrates example embodiments where operation 2210 may include at least one additional operation. Additional operations may include operation 2302, operation 2304, and/or operation 2306.

Operation 2302 illustrates detecting with an exhaled breath analysis device at least one indication of bioactive agent use by an individual. For example, as shown in FIGS. 18 through 21, exhaled breath analysis detector module 2004 may detect with an exhaled breath analysis device at least one indication of bioactive agent use by an individual. In one embodiment, exhaled breath analysis detector module 2004 may detect an indication of an specific isotope with a breath test analyzer subsequent to an individual ingesting a bioactive agent including the specific isotope. A further example of this may be found in Katzman et al., U.S. Pat. No. 6,491,643, which is incorporated herein by reference. A further example including a personal computer breath analyzer may be found in Cranley et al., U.S. Pat. No. 6,609,068, which is incorporated herein by reference. In some instances, exhaled breath analysis detector module 2004 may include a computer processor and/or a breath analyzer.

Further, operation 2304 illustrates detecting with a wireless exhaled breath analysis device at least one indication of bioactive agent use by an individual. For example, as shown in FIGS. 18 through 21, wireless exhaled breath analysis detector module 2006 may detect with a wireless exhaled breath analysis device at least one indication of bioactive agent use by an individual. In one embodiment, wireless exhaled breath analysis detector module 2006 may detect an indication of alcohol use with a breathalyzer configured to wirelessly connect to a computer with a Bluetooth connection. A further example of a wireless exhaled breath analysis device may be found in Cranley et al., U.S. Pat. No. 6,609,068, which is incorporated herein by reference. A wireless connection may include radio frequency communication, microwave communication, and/or infrared communication. In another example, the wireless exhaled breath analysis device may be wirelessly coupled to a computing device using, for example, an IEEE 802.11 computer network and/or a Bluetooth wireless sensor network. In some instances, wireless exhaled breath analysis detector module 2006 may include a computer processor and/or wireless instrumentation, such as a Bluetooth transmitter and/or receiver.

Operation 2306 illustrates detecting with a bioactive agent-dispensing inhalation collar at least one indication of bioactive agent use by an individual. For example, as shown in FIGS. 18 through 21, bioactive agent use detector module 2008 may detect with a bioactive agent-dispensing inhalation collar at least one indication of bioactive agent use by an individual. A bioactive agent-dispensing inhalation collar may include a collar with, for example, means for dispensing a bioactive agent, such as a reservoir and/or an accompanying valve and spray nozzle. Additionally, means for dispensing a bioactive agent may include means for dispensing an aerosol, vapor, a powder (e.g. pulmicort and/or foradil), and/or a mist, such as a nebulizer, means for measuring and/or detecting a condition, such as blood oxygen level and/or body temperature, and/or means for processing information, such as a computer processor and/or computer memory. Further, a bioactive agent may be dispensed and/or dispersed in and/or include a surfactant. The bioactive agent-dispensing collar may include means for detecting a bioactive agent, such as an oxygen sensor and/or a gas sensor, such as an electric nose. A further example of an electric nose may be found in Guoliang et al., U.S. Pat. No. 6,411,905, which is incorporated herein by reference. In one embodiment, bioactive agent use detector module 2008 may detect an indication of a bioactive agent that gives a certain odor when ingested with a bioactive agent-dispensing inhalation collar fitted with an electric nose. Additionally, a bioactive agent-dispensing inhalation collar may include means for power, such as a battery and/or circuitry for receiving power from an external source, such as an AC adapter power supply. In some instances, bioactive agent use detector module 2008 may include a computer processor and/or means for detecting, such as a chemical sensor and/or a detector (the electric nose discussed above).

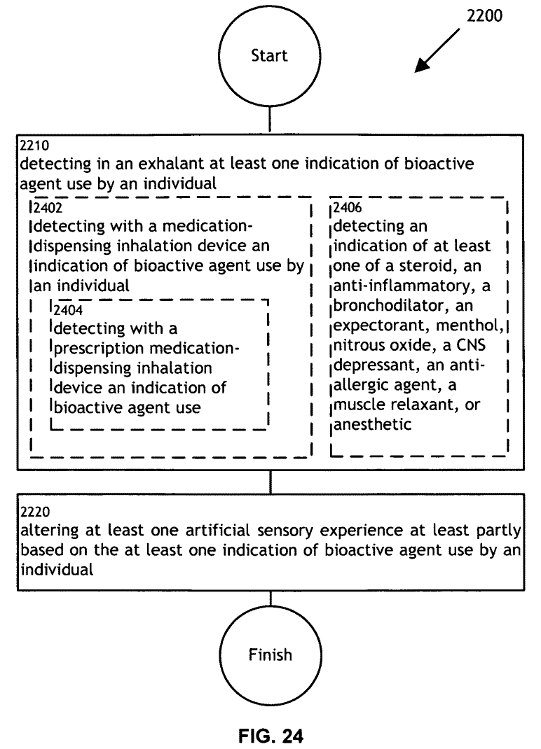

FIG. 24 illustrates alternative embodiments of the example operational flow 2200 of FIG. 22. FIG. 24 illustrates example embodiments where operation 2210 may include at least one additional operation. Additional operations may include operation 2402, operation 2404, and/or operation 2406.

Operation 2402 illustrates detecting with a medication-dispensing inhalation device an indication of bioactive agent use by an individual. For example, as shown in FIGS. 18 through 21, medication-dispensing device detector module 2010 may detect with a medication-dispensing inhalation device an indication of bioactive agent use by an individual. In one embodiment, medication-dispensing device detector module 2010 may detect with a medication-dispensing inhalation necklace adapted with a gas sensor an indication of the concentration of a by-product given off by an ingested bioactive agent. Additionally, a breath and/or gas may be analyzed by a detection device coupled to the medication-dispensing inhalation device, such as tubing delivering a gas (breath) sample to a gas chromatograph from the medication-dispensing inhalation device. A medication dispensing-inhalation device may include a device, such as a collar, a necklace, and or a bracelet configured to dispense medication, for example, with a bioactive agent reservoir and/or dispensing nozzles. In some instances, medication-dispensing device detector module 2010 may include a computer processor and/or means for detecting, such as a chemical sensor and/or a detector (e.g., the electric nose discussed above).

Further, operation 2404 illustrates detecting with a prescription medication-dispensing inhalation device an indication of bioactive agent use. For example, as shown in FIGS. 18 through 21, prescription medication-dispensing device detector module 2012 may detect with a prescription medication-dispensing inhalation device an indication of bioactive agent use. A prescription medication-dispensing inhalation device may include a device configured to dispense a medication only available from a licensed health care provider. Some examples of a prescription medication available from a licensed health care provider may include a bronchodilator (including beta-agonists and anti-cholinergics), such as albuterol, coricosteroids, nitrous oxide, a sedative, such as benzodiazepine, Theophylline, nedocromil sodium, fluticasone and salmeterol, and/or combinations thereof. In one embodiment, prescription medication-dispensing device detector module 2012 may detect an indication of a prescription medication-dispensing inhalation device configured for dispensing ciclesonide. The indication of the prescription medication-dispensing inhalation device may include, for example, an electrical and/or wireless signal from the device to a computer, computer software program, and/or computer monitor. In some instances, prescription medication-dispensing device detector module 2012 may include a computer processor.

Operation 2406 illustrates detecting an indication of at least one of a steroid, an anti-inflammatory, a bronchodilator, an expectorant, menthol, nitrous oxide, a CNS depressant, an anti-allergic agent, a muscle relaxant, or anesthetic. For example, as shown in FIGS. 18 through 21, bioactive agent detector module 2014 may detect an indication of at least one of a steroid, an anti-inflammatory, a bronchodilator, an expectorant, menthol, nitrous oxide, a CNS-depressant, an anti-allergenic agent, a muscle relaxant, or an anesthetic. One example of a steroid may include an anabolic steroid, which may be a derivative of androgens (such as testosterone), for stimulating growth. Another example of a steroid may include a corticosteroid, which may be often used as an anti-inflammatory prescribed for asthma. An anti-inflammatory may include a bioactive agent utilized to treat and/or reduce inflammation. Some examples of an anti-inflammatory may include glucocorticoids, ibuprofen, and/or naproxen. A bronchodilator may include a substance that dilates the bronchi and bronchioles decreasing airway resistance and thereby facilitating airflow. A bronchodilator may include a beta-agonist, an anti-cholinergic, and/or a muscle relaxant, such as theophylline. An expectorant may include a bioactive agent used for dissolving and/or bringing up mucus from the lungs, respiratory tract, and/or trachea. Some examples of an expectorant may include guaifenesin and/or tyloxapol. Menthol may include an organic and/or synthetic compound with local anesthetic and counterirritant qualities often used for relieving throat irritation and/or as a decongestant. Nitrous oxide may include a gas often used as a weak general anesthetic. A CNS-depressant, such as benzodiazepine and/or a sedative, may include one class of psychoactive drugs with varying hypnotic, sedative, anxiolytic, anticonvulsant, muscle relaxant and amnesic properties, which may be mediated by slowing down the central nervous system. In one embodiment, bioactive agent indication presenter module 2038 may detect an indication of a benzodiazepine. One example of benzodiazepine delivery through an inhalation route may be disclosed in Kim et al., U.S. Patent Publication No. 2003/0032638, which is incorporated herein by reference. An anti-allergic agent may include an agent configured to block the action of allergic mediators and/or to prevent activation of cells and degranulation processes. Some examples of an anti-allergic agent may include an antihistamine and/or cromones like mast cell stabilizers, such as cromoglicic acid and nedocromil sodium. A muscle relaxant may include a bioactive agent for affecting skeletal muscle function and/or decreasing muscle tone. One example of a muscle relaxant may include a methylxanthine, such as Theophylline. An anesthetic may include an inhalational general anesthetic, such as halothane, desflurane, enflurane, isoflurane, and/or sevoflurane. Detecting an indication of a bioactive agent may include discovering the presence of the bioactive agent, such as through a chemical testing means (e.g, a breathalyzer, a litmus test, and/or a drug test). In some instances, bioactive agent indication presenter module 2038 may include a computer processor, a printer, a display, and/or a mobile device.

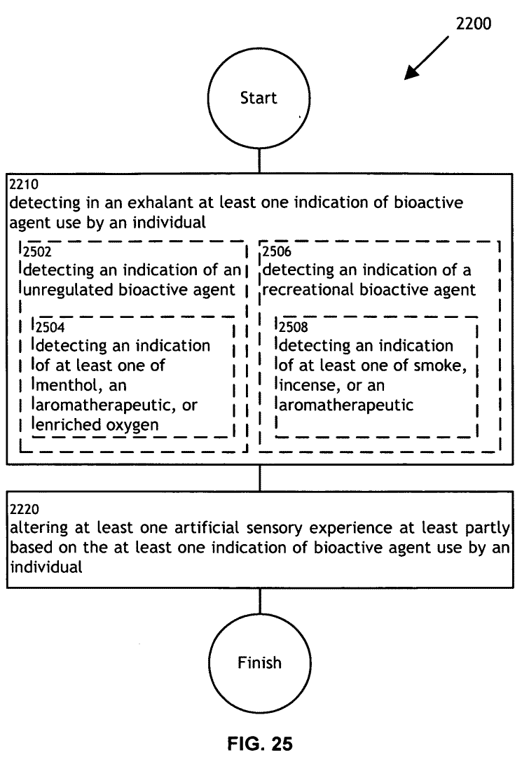

FIG. 25 illustrates alternative embodiments of the example operational flow 2200 of FIG. 22. FIG. 25 illustrates example embodiments where operation 2210 may include at least one additional operation. Additional operations may include operation 2502, operation 2504, operation 2506, and/or operation 2508.

Operation 2502 illustrates detecting an indication of an unregulated bioactive agent. For example, as shown in FIGS. 18 through 21, unregulated bioactive agent detector module 2016 may detect an indication of an unregulated bioactive agent. In one embodiment, unregulated bioactive agent detector module 2016 may accept an indication of enriched oxygen. Some examples of an unregulated bioactive agent may include enriched oxygen, aromas used for aromatherapy, and/or menthol. In another embodiment, unregulated bioactive agent detector module 2016 may accept an indication of an aromatherapeutic, such as lavender oil. In some instances, unregulated bioactive agent detector module 2016 may include a computer processor.

Further, operation 2504 illustrates detecting an indication of at least one of menthol, an aromatherapeutic, or enriched oxygen. For example, as shown in FIGS. 18 through 21, exhaled bioactive agent detector module 2018 may detect an indication of at least one of menthol, an aromatherapeutic, or enriched oxygen. In one embodiment, exhaled bioactive agent detector module 2018 may detect an indication of menthol. Menthol may include an organic and/or synthetic compound with local anesthetic and counterirritant qualities often used for relieving throat irritation and/or as a decongestant. Aromatherapy may include the use of an aromatherapeutic, which may include a volatile material, such as an essential oil. Some examples of an aromatherapeutic may include essential oils (eucalyptus oil and/or grapefruit oil), absolutes (jasmine and/or rose absolute), herbal distillates (lemon balm and/or chamomile), and/or a volatile medication, such as a decongestant with menthol. The volatile material may be applied using aerial diffusion, direct inhalation, and/or a topical application. Enriched oxygen may be used as a mild euphoric and/or a performance booster, for example, during an individual's exercise session while experiencing a virtual world, e.g., while playing Wii fit. In some instances, exhaled bioactive agent detector module 2018 may include a computer processor and/or a detection device, such as an oxygen sensor and/or a gas chromatograph.

Operation 2506 illustrates detecting an indication of a recreational bioactive agent. For example, as shown in FIGS. 18 through 21, recreational bioactive agent detector module 2020 may detect an indication of a recreational bioactive agent. In one embodiment, recreational bioactive agent detector module 2020 may detect an indication of an aromatherapeutic. Some other examples of a recreational inhalation agent may include smoke (for example, simulating proximity to a campfire) and/or a scent, for example, using a scent in a virtual scent environment, such as a learning environment (e.g., cooking and/or chemistry). Another example of a recreational inhalation agent may include an inhalation agent for a ceremonial purpose, such as tobacco smoke. In some instances, recreational bioactive agent detector module 2020 may include a computer processor and/or a detection device, such as an oxygen sensor and/or a gas chromatograph.

Further, operation 2508 illustrates detecting an indication of at least one of smoke, incense, or an aromatherapeutic. For example, as shown in FIGS. 18 through 21, recreational bioactive agent detector module 2020 may detect an indication of at least one of smoke, incense, or an aromatherapeutic. In one embodiment, recreational bioactive agent detector module 2020 may detect an indication of smoke. Smoke may include the collection of airborne colloids. Some uses of smoke may include simulation of a campfire and/or the use of smoke in a ritual when incense, sage, and/or a resin are burned to produce a smell for a spiritual purpose. Incense may include an aromatic biotic material and/or the smoke released when the aromatic biotic material is burned. Incense may be used for religious, practical, and/or aesthetic purposes. An aromatherapeutic may include a volatile material, such as an essential oil. Some examples of an aromatherapeutic may include essential oils (eucalyptus oil and/or grapefruit oil), absolutes (jasmine and/or rose absolute), herbal distillates (lemon balm and/or chamomile), and/or a volatile medication, such as a decongestant with menthol. The volatile material may be applied using aerial diffusion, direct inhalation, and/or a topical application. In another embodiment, recreational bioactive agent detector module 2020 may detect incense with a smoke detector. In some instances, recreational bioactive agent detector module 2020 may include a computer processor and/or detection means, such as an oxygen detector and/or a smoke detector.

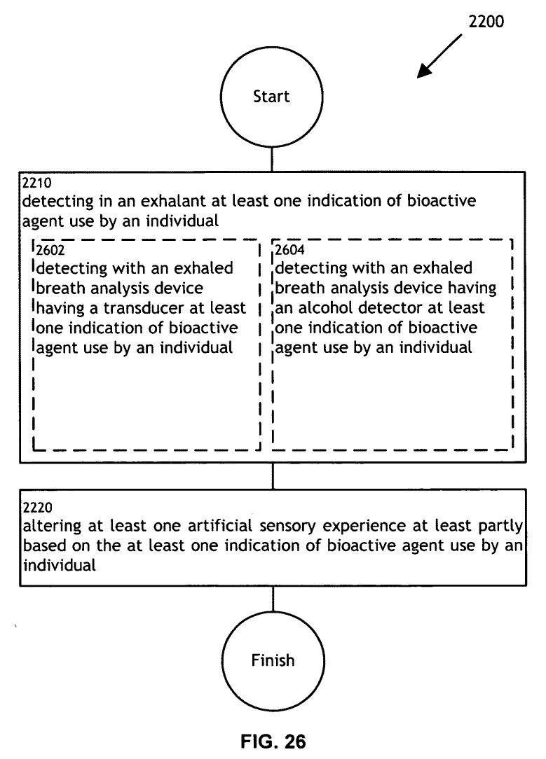

FIG. 26 illustrates alternative embodiments of the example operational flow 2200 of FIG. 22. FIG. 26 illustrates example embodiments where operation 2210 may include at least one additional operation. Additional operations may include operation 2602, and/or operation 2604.

Operation 2602 illustrates detecting with an exhaled breath analysis device having a transducer at least one indication of bioactive agent use by an individual. For example, as shown in FIGS. 18 through 21, transducer device detector module 2022 may detect with an exhaled breath analysis device having a transducer at least one indication of bioactive agent use by an individual. In one embodiment, transducer device detector module 2022 may detect with a breathalyzer having a transducer an indication of menthol use by an individual. One example of an exhaled breath analysis device with a transducer may be found in Pikul, U.S. Pat. No. 3,946,726, which is incorporated herein by reference. Another example of an alcohol breathalyzer with a transducer may be found in Porter et al., which is incorporated herein by reference. In some instances, transducer device detector module 2022 may include a computer processor and/or means for detection, such as a breathalyzer and/or a smoke detector.

Operation 2604 illustrates detecting with an exhaled breath analysis device having an alcohol detector at least one indication of bioactive agent use by an individual. For example, as shown in FIGS. 18 through 21, alcohol detector module 2024 may detect with an exhaled breath analysis device having an alcohol detector at least one indication of bioactive agent use by an individual. In one embodiment, alcohol detector module 2024 may detect use of menthol with a collar fitted with a breathalyzer. Some examples of an alcohol detector may include a Breathalyzer, which detects by utilizing photocells to measure a color change associated with a dichromate and alcohol reaction, an Intoxilyzer, which utilizes infrared spectroscopy, and/or an Alcosensor, which utilizes a fuel cell. In some instances, alcohol detector module 2024 may include a computer processor and or an alcohol detector.

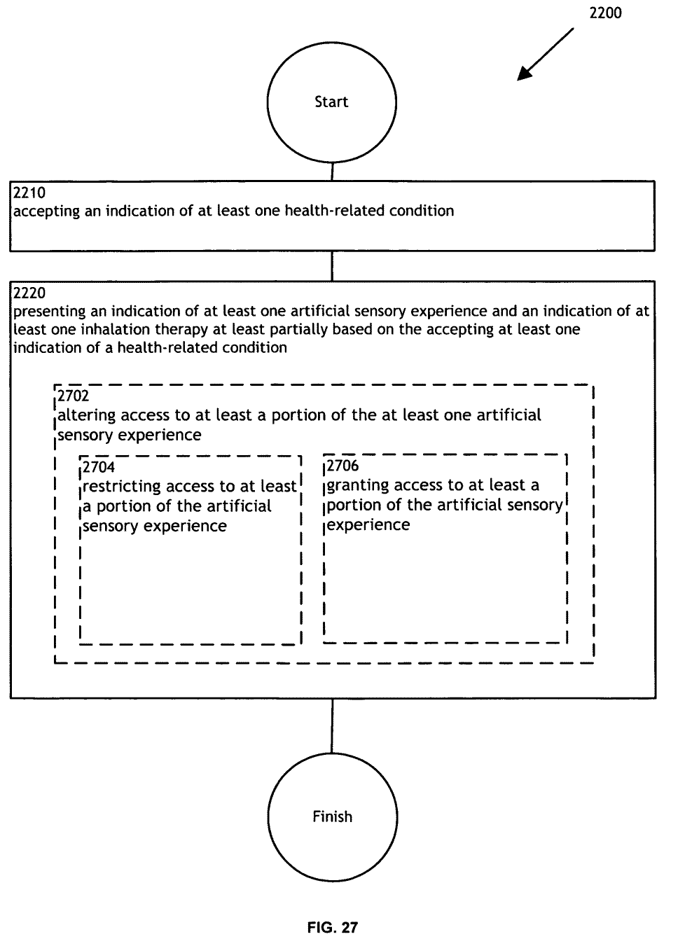

FIG. 27 illustrates alternative embodiments of the example operational flow 2200 of FIG. 22. FIG. 27 illustrates example embodiments where operation 2220 may include at least one additional operation. Additional operations may include operation 2702, operation 2704, and/or operation 2706.

Operation 2702 illustrates altering access to at least a portion of the at least one artificial sensory experience. For example, as shown in FIGS. 18 through 21, access alterer module 2102 may alter access to at least a portion of the artificial sensory experience, for example, to modify at least one effect of the bioactive agent. In one instance, access alterer module 2102 may alter access to a portion of an artificial sensory experience including a photo gallery portion of a social network website to modify at least one effect of the bioactive agent, for example, an antidepressant. Such altered access may function therapeutically to prevent access of an individual to potentially depressing, stressful, or otherwise triggering sensory experiences, and/or the modified access may involve presentation of a sensory experience that affirmatively improves a condition (e.g., bright sunny images for a clinically depressed individual). In some instances, access alterer module 2102 may include a computer processor.

Further, operation 2704 illustrates restricting access to at least a portion of the artificial sensory experience. For example, as shown in FIGS. 18 through 21, restricter module 2104 may restrict access to at least a portion of the artificial sensory experience. In one instance, restricter module 2104 may restrict access to a portion of an artificial sensory experience including a virtual world designed to overcome a flying phobia, where access to a portion of a simulated flying experience is restricted including a jet take-off portion. In some instances, restricter module 2104 may include a computer processor.

Further, operation 2706 illustrates granting access to at least a portion of the artificial sensory experience. For example, as shown in FIGS. 18 through 21, granter module 2106 may grant access to at least a portion of the artificial sensory experience. In one instance and continuing with the above example, granter module 2106 may grant access to at least a portion of a virtual world designed to overcome a flying phobia, where access to a portion of a simulated flying experience is granted including a jet landing portion. Such a simulation with gradually increasing contact with the object of the fear may serve to provide conditioning for the individual to eventually overcome the phobia. In some instances, granter module 2106 may include a computer processor.

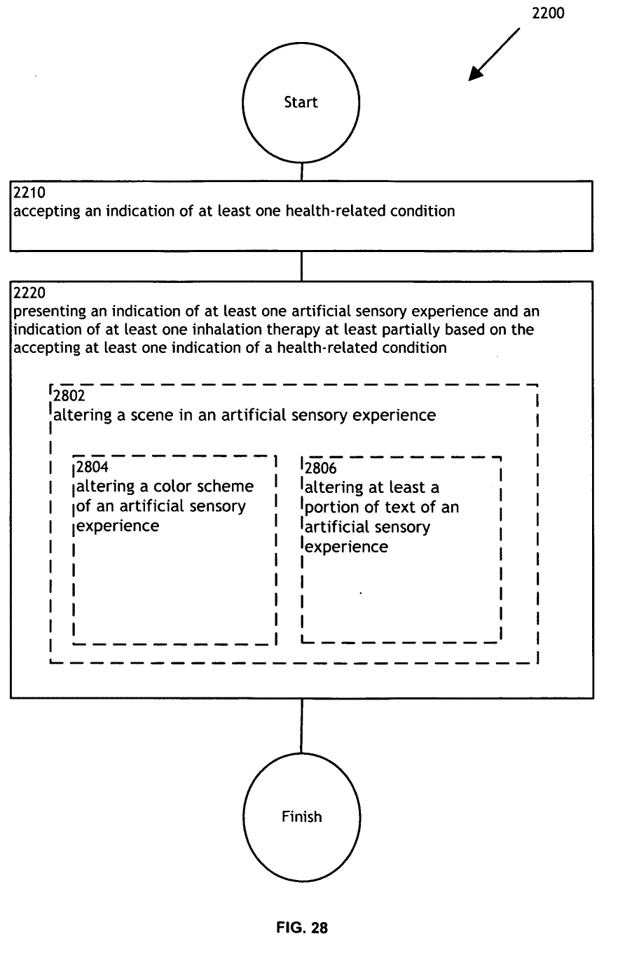

FIG. 28 illustrates alternative embodiments of the example operational flow 2200 of FIG. 22. FIG. 28 illustrates example embodiments where operation 2220 may include at least one additional operation. Additional operations may include operation 2802, operation 2804, and/or operation 2806.

Operation 2802 illustrates altering a scene in an artificial sensory experience. For example, as shown in FIGS. 18 through 21, scene alterer module 2108 may alter a scene in an artificial sensory experience, for example, to modify at least one effect of the bioactive agent. In one instance and continuing with the above example, scene alterer module 2108 may alter a visual object, such as adding window covers, in a virtual world designed to overcome a flying phobia to modify at least one effect of an inhaled anti-anxiety medication. In this example, the window covers may reduce anxiety experienced by the individual in addition to anxiety reduction mediated by the anti-anxiety medication. Additional examples of a scene and/or visual object may include a virtual character, an action performed by the virtual character, character artifacts, such as weapons, clothing, and/or tools, illumination, focus, magnification, dodging (altering), and/or apparent speed. In some instances, scene alterer module 2108 may include a computer processor.

Further, operation 2804 illustrates altering a color scheme of an artificial sensory experience. For example, as shown in FIGS. 18 through 21, color scheme alterer module 2110 may alter a color scheme of an artificial sensory experience, for example, to modify at least one effect of the bioactive agent. In one instance, color scheme alterer module 2110 may alter a color scheme by adding brighter background lights and colors in a virtual world designed to overcome depression to alter an effect of an anti-depression medication. Such a color scheme modification may help to overcome depression, seasonal affective disorder, and/or other disorders because it has been purported that color and/or light may affect nonvisual psychological processes. Discussion regarding the effects of color and/or light on nonvisual psychological processes may be found in Knez, Effects of colour of light on nonvisual psychological processes, JOURNAL OF ENVIRONMENTAL PSYCHOLOGY, 21(2):201-08 (2001); M. R Basso Jr., Neurobiological relationships between ambient lighting and the startle response to acoustic stress in humans, INT J NEUROSCI., 110(3-4):147-57 (2001), and Lam et al., The Can-SAD Study: a randomized controlled trial of the effectiveness of light therapy and fluoxetine in patients with winter seasonal affective disorder, AMERICAN JOURNAL OF PSYCHIATRY, 163(5):805-12 (2006), each incorporated by reference. In some instances, color scheme alterer module 2110 may include a computer processor.

Further, operation 2806 illustrates altering at least a portion of text of an artificial sensory experience. For example, as shown in FIGS. 18 through 21, text alterer module 2112 may alter at least a portion of text of an artificial sensory experience, for example, to modify at least one effect of the bioactive agent. In one instance, text alterer module 2112 may alter a portion of instructional text in a virtual world including a computer game to alter an effect of a bioactive agent including a prescribed herbal memory supplement. Text modification may improve memory by utilizing techniques such as underlining, highlighting, boldfacing, and/or mnemonics as discussed in Carney, R. N., & Levin, J. R., Mnemonic instruction with a focus on transfer, JOURNAL OF EDUCATIONAL PSYCHOLOGY, 92(4):783-90, which is incorporated herein by reference. Another example may include instructional text providing contextual or associative information, perhaps individualized, to aid in remembering during the rest of a module. Another example of text modification and memory may include modifying the use of interactive components, e.g. via a keyboard and/or speakers, to use multiple forms of memory input, including visual, auditory, motor, and contextual. For example, this may be used to aid memory and/or in learning disorders such as dysgraphia, and/or memory disorders, such as in conjunction with memory-enhancing medications, for example cholinesterase inhibitors or herbal memory supplements. Additionally, text messages may be added and/or altered based on cognitive therapy but individualized for the person, affliction, and/or medication (e.g. an antidepressant and instructions to work toward a goal within a game that will aid in refuting automatic negative thoughts). In some instances, text alterer module 2112 may include a computer processor.

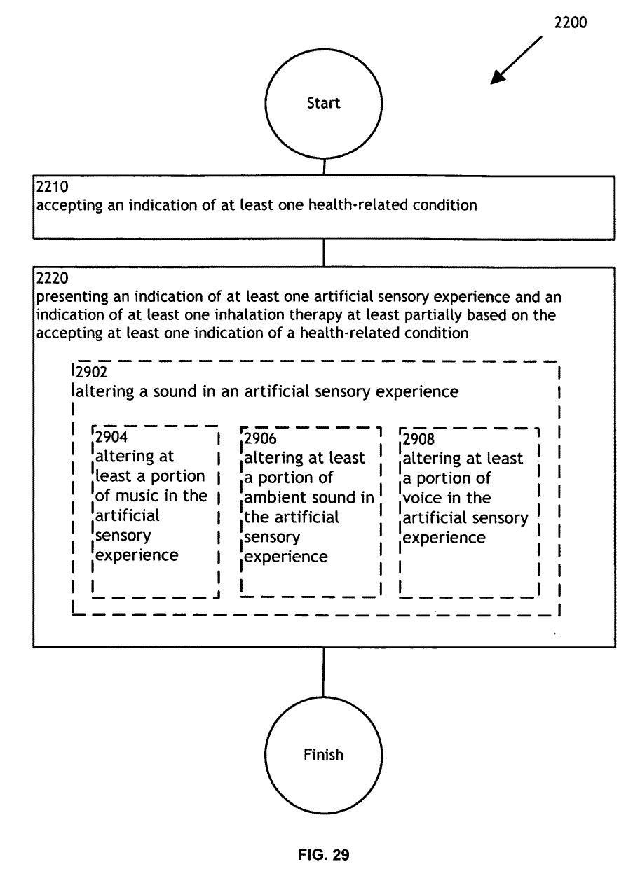

FIG. 29 illustrates alternative embodiments of the example operational flow 2200 of FIG. 22. FIG. 29 illustrates example embodiments where operation 2220 may include at least one additional operation. Additional operations may include operation 2902, operation 2904, operation 2906, and/or operation 2908.

Operation 2902 illustrates altering a sound in an artificial sensory experience. For example, as shown in FIGS. 18 through 21, sound alterer module 2114 may alter a sound in an artificial sensory experience. In one instance, sound alterer module 2114 may alter a sound in a virtual world including an instructor's voice tone in an instructional tutorial. This may be done as a custom-tailored feature. For example, various voice tones may be tested with an individual in order to find one that has the most significant benefit for the individual, in conjunction with the bioactive agent. In some instances, sound alterer module 2114 may include a computer processor.

Further, operation 2904 illustrates altering at least a portion of music in the artificial sensory experience. For example, as shown in FIGS. 18 through 21, music alterer module 2116 may alter at least a portion of music in the artificial sensory experience. In one instance, music alterer module 2116 may alter a portion of music including background music in an instructional tutorial. Music in the artificial sensory experience may include pitch, rhythm, tempo, meter, and articulation, dynamics, lyrics, timbre and texture. In one specific instance, music alterer module 2116 may alter a portion of uptempo music to soothing classical music in an artificial sensory experience coupled with administration of an anxiolytic. Such a music alteration may serve to provide a calming and/or relaxing environment where the effects of the anxiolytic may be facilitated and/or enhanced. In another instance, a sound pitch may be altered to affect bone (as in healing fractures and/or promoting bone growth) and/or sinuses (including joints). Additionally, music alterer module 2116 may include providing another type of sound, such as a low frequency, to aid in healing, e.g. in conjunction with pain medication and/or an anti-inflammatory medication. In another example, the sound may originate from a natural source, for instance a purr of a cat, possibly provided at a particular pitch, to aid in relaxation, as in conjunction with a tranquilizer, and/or in healing tissue in conjunction with pain medication or anti-inflammatories. Further discussion regarding low frequency therapeutic biomechanical stimulation may be found in von Muggenthaler, E. K., The Felid purr: low frequency therapeutic biomechanical stimulation, 12th International Conference on Low Frequency Noise and Vibration and its Control, Bristol, UK, Sep. 18-20, 2006, Abstract located at Fauna Communications Research Institute <http://animalvoice.com/catpurrP.htm#2pAB7.%20The %20felid%20purr:%20A%20healing%20mechanism?%20Session:%20Tuesday%20Afternoon,%20Dec%2004%20Time:%203:15>, and Simos et al., U.S. patent application Ser. No. 11/262,884, each incorporated herein by reference. In some instances, music alterer module 2116 may include a computer processor.

Further, operation 2906 illustrates altering at least a portion of ambient sound in the artificial sensory experience. For example, as shown in FIGS. 18 through 21, ambient sound alterer module 2118 may alter at least a portion of ambient sound in the artificial sensory experience. In one instance, ambient sound alterer module 2118 may alter the ambient noise in an artificial sensory experience including a level of white noise in the online virtual world Second Life. Ambient sound and/or noise may include white noise, background noise, such as people talking or sounds naturally occurring in nature, and/or room noise. Changing the level of white noise may, for example, enhance the effect of an attention deficit drug such as Ritalin, or it may enhance the sedative properties of a steep medication or tranquilizer. Further discussion of the effects of white noise may be found in Spencer, J. A. et al., White noise and sleep induction, ARCH DIS CHILD 65(1):135-37 (1990), which is incorporated by reference. In some instances, ambient sound alterer module 2118 may include a computer processor.

Further, operation 2908 illustrates altering at least a portion of voice in the artificial sensory experience. For example, as shown in FIGS. 18 through 21, voice alterer module 2120 may alter at least a portion of voice in the artificial sensory experience. In one instance, voice alterer module 2120 may alter a voice rhythm in an online tutorial. Such alteration may enhance the effect of an inhaled attention deficit medication, for example by elimination or reduction of monotonic qualities in the voice rhythm of the online tutorial, for example. Some examples of a voice may include a voice recording, an artificially generated voice, and/or a human voice. In some instances, voice alterer module 2120 may include a computer processor.

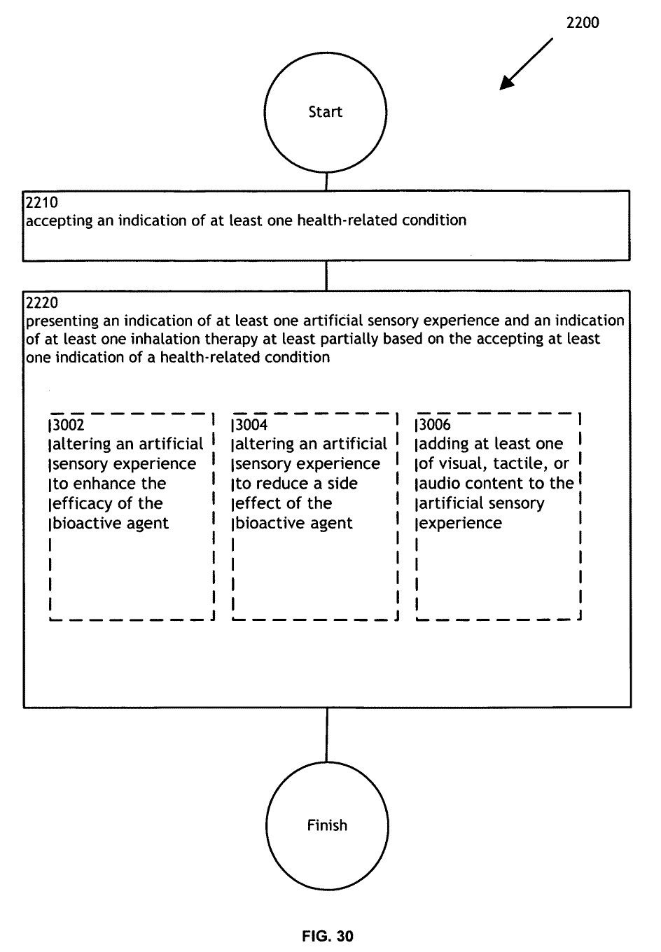

FIG. 30 illustrates alternative embodiments of the example operational flow 2200 of FIG. 22. FIG. 30 illustrates example embodiments where operation 2220 may include at least one additional operation. Additional operations may include operation 3002, operation 3004, and/or operation 3006.

Operation 3002 illustrates altering an artificial sensory experience to enhance the efficacy of the bioactive agent. For example, as shown in FIGS. 18 through 21, efficacy alterer module 2122 may alter an artificial sensory experience to enhance the efficacy of the bioactive agent. In one instance, efficacy atterer module 2122 may modify a virtual world by adding uptempo music to enhance the efficacy of an inhaled antidepressant. Further discussion of music effects may be found in Schellenberg, E. G. et al., Exposure to music and cognitive performance: tests of children and adults, PSYCHOLOGY OF MUSIC, Vol. 35, No. 1, 5-19 (2007), which is incorporated herein by reference. In some instances, efficacy alterer module 2122 may include a computer processor.

Operation 3004 illustrates altering an artificial sensory experience to reduce a side effect of the bioactive agent. For example, as shown in FIGS. 18 through 21, side effect reduction alterer module 2124 may alter an artificial sensory experience to reduce a side effect of the bioactive agent. In one instance, side effect reduction alterer module 2124 may modify a virtual world by adding music and/or sounds occurring in nature for reducing a side effect including a headache due to an administration of penicillin. Further discussion of music effects upon a side effect may be found in Siedliecki, S. L. and Good, M., Effect of music on power, pain, depression and disability, JOURNAL OF ADVANCED NURSING 54(5):553-62 (2006), and Natural distractions reduce pain—study finds that sights and sounds of nature aid in pain reduction—Brief Article, MEN'S FITNESS. October 2001, each incorporated by reference. In some instances, side effect reduction alterer module 2124 may include a computer processor.

Operation 3006 illustrates adding at least one of visual, tactile, or audio content to the artificial sensory experience. For example, as shown in FIGS. 18 through 21, adder module 2126 may add at least one of visual, tactile, or audio content to the artificial sensory experience. In one instance, adder module 2126 may add audio content including calming music to an artificial sensory experience including a virtual world for treating a phobia of heights. The adding function may include increasing, creating, and/or combining content. Some examples of visual content may include visual objects, light amount and/or intensity, and/or color schemes. Some examples of tactile content may include a breeze and/or falling water drops (e.g., to simulate rain). Examples of audio content may include music, voices, artificial sounds, and/or white noise. In some instances, adder module 2126 may include a computer processor.

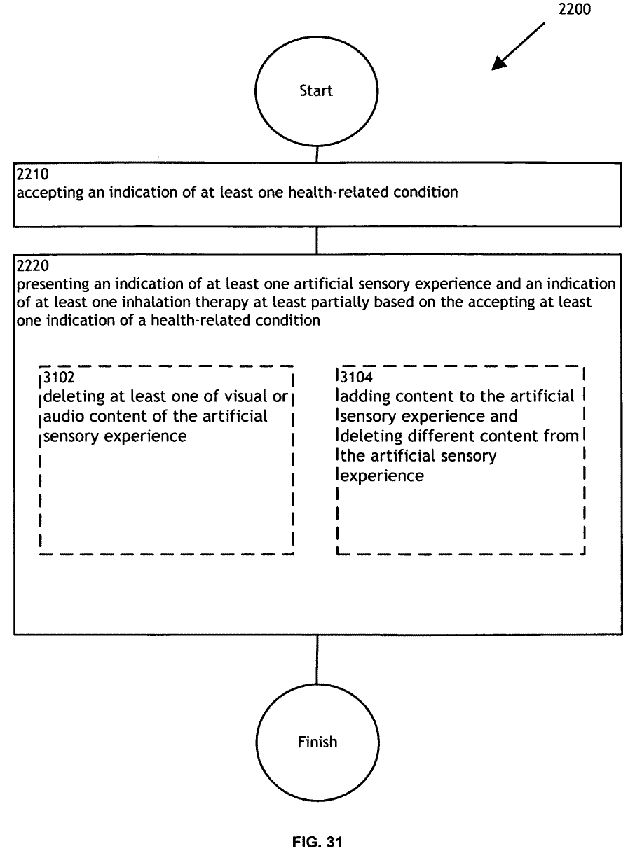

FIG. 31 illustrates alternative embodiments of the example operational flow 2200 of FIG. 22. FIG. 31 illustrates example embodiments where operation 2220 may include at least one additional operation. Additional operations may include operation 3102, and/or operation 3104.

Operation 3102 illustrates deleting at least one of visual or audio content of the artificial sensory experience. For example, as shown in FIGS. 18 through 21, deleter module 2128 may delete at least one of visual or audio content of the artificial sensory experience. In one instance, deleter module 2128 may delete visual content including a bright lighting environment in a virtual world for enhancing the effect of a medication for migraine headache. Deleting may include reducing and/or eliminating visual and/or audio content. In some instances, deleter module 2128 may include a computer processor.

Operation 3104 illustrates adding content to the artificial sensory experience and deleting different content from the artificial sensory experience. For example, as shown in FIGS. 18 through 21, adder module 2126 may add content to the artificial sensory experience and deleter module 2128 may delete different content from the artificial sensory experience. In one instance, adder module 2126 may add classical background music to a virtual world and deleter module 2128 may delete ambient street noise, for example, using sound detection and/or noise-cancellation technology, to enhance the effect of a sedative or other similar bioactive agent. In some instances, adder module 2126 and/or deleter module 2128 may include a computer processor.

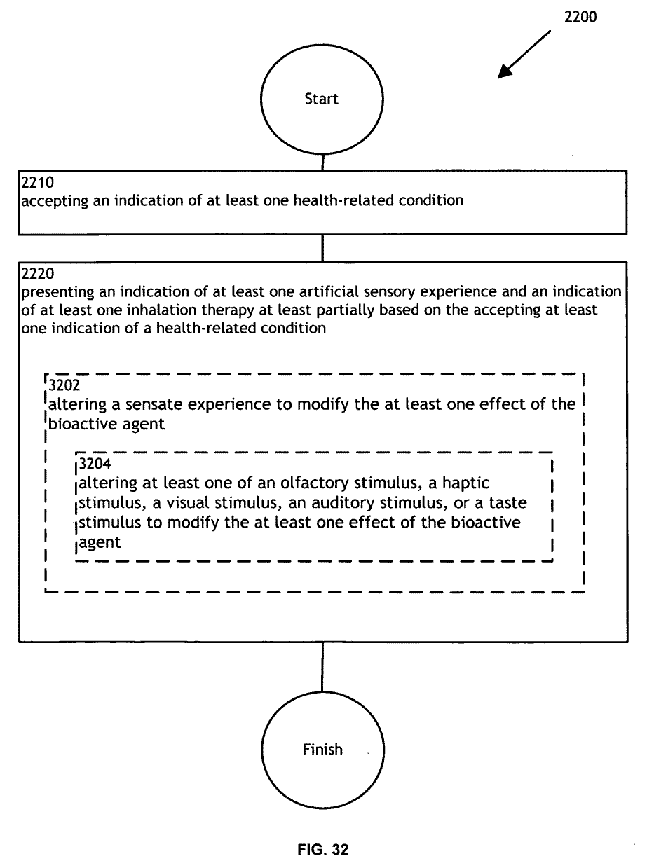

FIG. 32 illustrates alternative embodiments of the example operational flow 2200 of FIG. 22. FIG. 32 illustrates example embodiments where operation 2220 may include at least one additional operation. Additional operations may include operation 3202, and/or operation 3204.

Operation 3202 illustrates altering a sensate experience to modify the at least one effect of the bioactive agent. For example, as shown in FIGS. 18 through 21, sensate alterer module 2130 may alter a sensate experience to modify the at least one effect of the bioactive agent. In one instance, sensate alterer module 2130 may modify a sensate experience including adding an aroma to enhance the effect of an anxiolytic drug or other similar bioactive agent. A sensate experience may include a thing perceived by the senses, such as an aroma, a sound, a feel, a taste, and/or a sight. In some instances, sensate alterer module 2130 may include a computer processor.

Further, operation 3204 illustrates altering at least one of an olfactory stimulus, a haptic stimulus, a visual stimulus, an auditory stimulus, or a taste stimulus to modify the at least one effect of the bioactive agent. For example, as shown in FIGS. 18 through 21, stimulus alterer module 2132 may alter at least one of an olfactory stimulus, a haptic stimulus, a visual stimulus, an auditory stimulus, or a taste stimulus to modify the at least one effect of the bioactive agent. In one instance, stimulus alterer module 2132 may alter an olfactory stimulus by adding a floral aroma and/or gentle vibration to enhance a relaxing effect of a sedative or other similar bioactive agent, such as an antianxiety medication. Further discussion of an olfactory stimulus may be found in Shaw, D. et al., Anxiolytic effects of lavender oil inhalation on open-field behaviour in rats, PHYTOMEDICINE, 14(9):613-20 (2007), incorporated by reference. In another embodiment, stimulus alterer module 2132 may alter a visual stimulus including a light intensity setting in a virtual world. In this embodiment, light intensity in the virtual world may be dimmed, for example, to create a relaxing environment for an anxiety sufferer. In some instances, stimulus alterer module 2132 may include a computer processor.

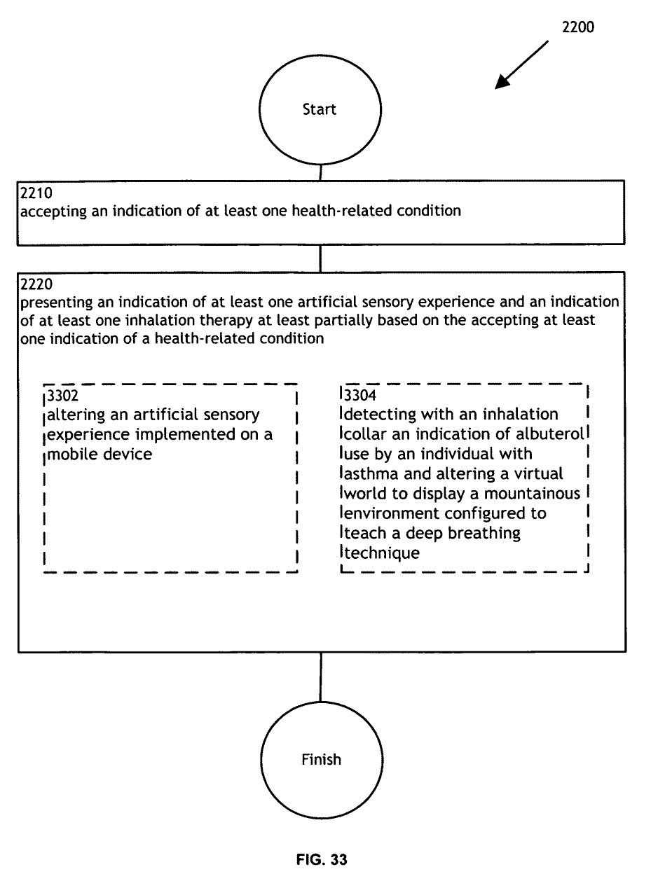

FIG. 33 illustrates alternative embodiments of the example operational flow 2200 of FIG. 22. FIG. 33 illustrates example embodiments where operation 2220 may include at least one additional operation. Additional operations may include operation 3302, and/or operation 3304.

Operation 3302 illustrates altering an artificial sensory experience implemented on a mobile device. For example, as shown in FIGS. 18 through 21, mobile device alterer module 2134 may alter an artificial sensory experience implemented on a mobile device. In one instance, mobile device alterer module 2134 may modify a virtual world implemented in a web browser on a laptop computer having wireless capability and a battery by changing a background color theme to a brighter color theme in the virtual world. An artificial sensory experience alteration, such as the color change in the above example, may enhance the effect of a bioactive agent. For example, creating a more pleasant environment in the artificial sensory experience by altering the color theme in the above example may enhance the anti-depressant drug effect while an anti-depressant is bioavailable. Some examples of a mobile device may include a laptop or notebook computer, a personal digital assistant (PDA), an ipod, a smartphone, an Enterprise digital assistant (EDA), and/or a pager. In another example, mobile device alterer module 2134 may modify a city image by providing a soothing image having fewer people in the same part of the city and combining the modified image with an anti-anxiety medicine for alleviating a phobia, such as agoraphobia. In another example, mobile device alterer module 2134 may provide a stepwise procedure, with a gradually less specific procedure and/or less steps, for a compulsive patient to follow to achieve a goal for a particular outing while taking a selective serotonin reuptake inhibitor (SSRI). In some instances, mobile device alterer module 2134 may include a computer processor.

Operation 3304 illustrates detecting with an inhalation collar an indication of albuterol use by an individual with asthma and altering a virtual world to display a mountainous environment configured to teach a deep breathing technique. For example, as shown in FIGS. 18 through 21, detector module 2002 and alterer module 2026 may detect with an inhalation collar an indication of albuterol use by an individual with asthma and alter a virtual world to display a mountainous environment configured to teach a deep breathing technique. In some instances, detector module 2002 may include a computer processor and/or an input device, such as a keyboard and/or detection instrumentation (e.g., a breathalyzer). In some instances, alterer module 2026 may include a computer processor.

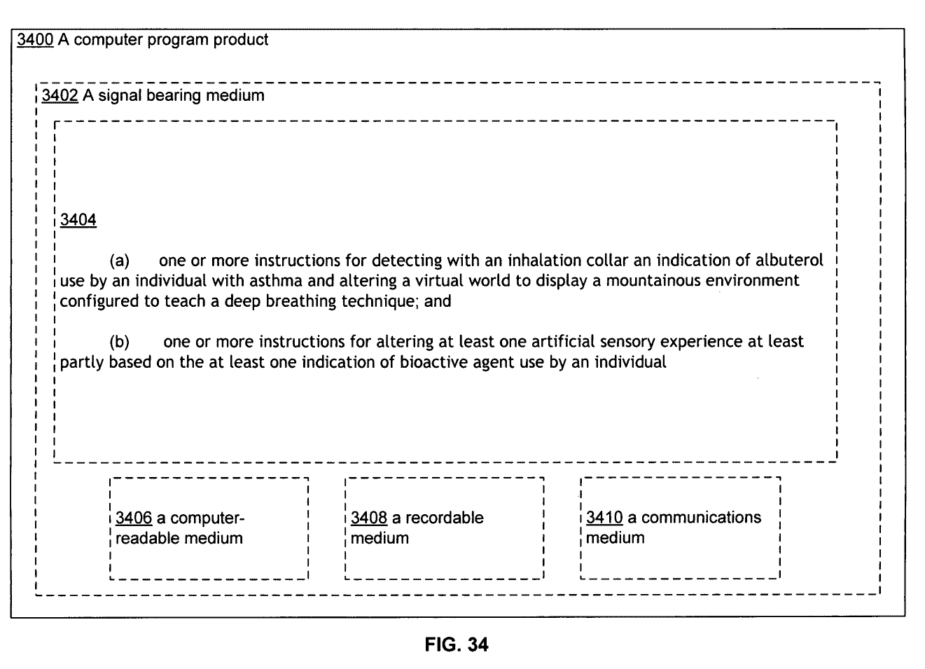

FIG. 34 illustrates a partial view of an example computer program product 3400 that includes a computer program 3404 for executing a computer process on a computing device. An embodiment of the example computer program product 3400 is provided using a signal-bearing medium 3402, and may include one or more instructions for detecting in an exhalant at least one indication of bioactive agent use by an individual and one or more instructions for altering at least one artificial sensory experience at least partly based on the at least one indication of bioactive agent use by an individual. The one or more instructions may be, for example, computer executable and/or logic-implemented instructions. In one implementation, the signal-bearing medium 3402 may include a computer-readable medium 3406. In one implementation, the signal bearing medium 3402 may include a recordable medium 3408. In one implementation, the signal bearing medium 3402 may include a communications medium 3410.

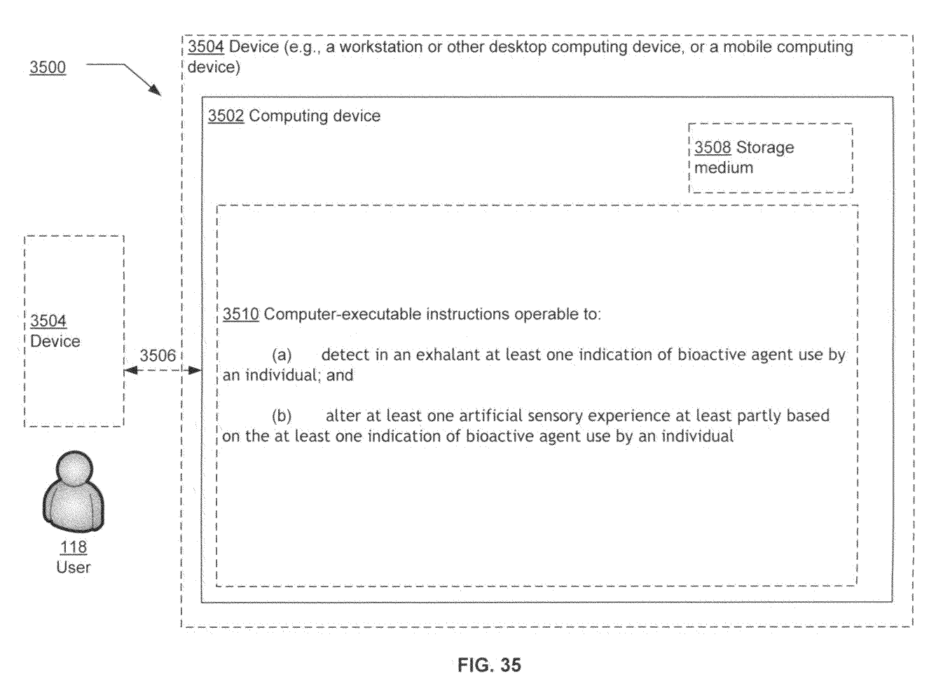

FIG. 35 illustrates an example system 3500 in which embodiments may be implemented. The system 3500 includes a computing system environment. The system 3500 also illustrates the user 118 using a device 3504, which is optionally shown as being in communication with a computing device 3502 by way of an optional coupling 3506. The optional coupling 3506 may represent a local, wide-area, or peer-to-peer network, or may represent a bus that is internal to a computing device (e.g., in example embodiments in which the computing device 3502 is contained in whole or in part within the device 3504). A storage medium 3508 may be any computer storage media.

The computing device 3502 includes computer-executable instructions 3510 that when executed on the computing device 3502 cause the computing device 3502 to detect in an exhalant at least one indication of bioactive agent use by an individual and alter at least one artificial sensory experience at least partly based on the at least one indication of bioactive agent use by an individual. As referenced above and as shown in FIG. 35, in some examples, the computing device 3502 may optionally be contained in whole or in part within the device 3504.

In FIG. 35, then, the system 3500 includes at least one computing device (e.g., 3502 and/or 3504). The computer-executable instructions 3510 may be executed on one or more of the at least one computing device. For example, the computing device 3502 may implement the computer-executable instructions 3510 and output a result to (and/or receive data from) the computing device 3504. Since the computing device 3502 may be wholly or partially contained within the computing device 3504, the device 3504 also may be said to execute some or all of the computer-executable instructions 3510, in order to be caused to perform or implement, for example, various ones of the techniques described herein, or other techniques.

The device 3504 may include, for example, a portable computing device, workstation, or desktop computing device. In another example embodiment, the computing device 3502 is operable to communicate with the device 3504 associated with the user 118 to receive information about the input from the user 118 for performing data access and data processing and presenting an output of the user-health test function at least partly based on the user data.

Although a user 118 is shown/described herein as a single illustrated figure, those skilled in the art will appreciate that a user 118 may be representative of a human user, a robotic user (e.g., computational entity), and/or substantially any combination thereof (e.g., a user may be assisted by one or more robotic agents). In addition, a user 118, as set forth herein, although shown as a single entity may in fact be composed of two or more entities. Those skilled in the art will appreciate that, in general, the same may be said of “sender” and/or other entity-oriented terms as such terms are used herein.

Following are a series of flowcharts depicting implementations. For ease of understanding, the flowcharts are organized such that the initial flowcharts present implementations via an example implementation and thereafter the following flowcharts present alternate implementations and/or expansions of the initial flowchart(s) as either sub-component operations or additional component operations building on one or more earlier-presented flowcharts. Those having skill in the art will appreciate that the style of presentation utilized herein (e.g., beginning with a presentation of a flowchart(s) presenting an example implementation and thereafter providing additions to and/or further details in subsequent flowcharts) generally allows for a rapid and easy understanding of the various process implementations. In addition, those skilled in the art will further appreciate that the style of presentation used herein also lends itself well to modular and/or object-oriented program design paradigms.

Those skilled in the art will appreciate that the foregoing specific exemplary processes and/or devices and/or technologies are representative of more general processes and/or devices and/or technologies taught elsewhere herein, such as in the claims filed herewith and/or elsewhere in the present application.

Those having skill in the art will recognize that the state of the art has progressed to the point where there is little distinction left between hardware, software, and/or firmware implementations of aspects of systems; the use of hardware, software, and/or firmware is generally (but not always, in that in certain contexts the choice between hardware and software can become significant) a design choice representing cost vs. efficiency tradeoffs. Those having skill in the art will appreciate that there are various vehicles by which processes and/or systems and/or other technologies described herein can be effected (e.g., hardware, software, and/or firmware), and that the preferred vehicle will vary with the context in which the processes and/or systems and/or other technologies are deployed. For example, if an implementer determines that speed and accuracy are paramount, the implementer may opt for a mainly hardware and/or firmware vehicle; alternatively, if flexibility is paramount, the implementer may opt for a mainly software implementation; or, yet again alternatively, the implementer may opt for some combination of hardware, software, and/or firmware. Hence, there are several possible vehicles by which the processes and/or devices and/or other technologies described herein may be effected, none of which is inherently superior to the other in that any vehicle to be utilized is a choice dependent upon the context in which the vehicle will be deployed and the specific concerns (e.g., speed, flexibility, or predictability) of the implementer, any of which may vary. Those skilled in the art will recognize that optical aspects of implementations with typically employ optically-oriented hardware, software, and or firmware.

In some implementations described herein, logic and similar implementations may include software or other control structures suitable to operation. Electronic circuitry, for example, may manifest one or more paths of electrical current constructed and arranged to implement various logic functions as described herein. In some implementations, one or more media are configured to bear a device-detectable implementation if such media hold or transmit a special-purpose device instruction set operable to perform as described herein. In some variants, for example, this may manifest as an update or other modification of existing software or firmware, or of gate arrays or other programmable hardware, such as by performing a reception of or a transmission of one or more instructions in relation to one or more operations described herein. Alternatively or additionally, in some variants, an implementation may include special-purpose hardware, software, firmware components, and/or general-purpose components executing or otherwise invoking special-purpose components. Specifications or other implementations may be transmitted by one or more instances of tangible transmission media as described herein, optionally by packet transmission or otherwise by passing through distributed media at various times.

Alternatively or additionally, implementations may include executing a special-purpose instruction sequence or otherwise invoking circuitry for enabling, triggering, coordinating, requesting, or otherwise causing one or more occurrences of any functional operations described above. In some variants, operational or other logical descriptions herein may be expressed directly as source code and compiled or otherwise invoked as an executable instruction sequence. In some contexts, for example, C++ or other code sequences can be compiled directly or otherwise implemented in high-level descriptor languages (e.g., a logic-synthesizable language, a hardware description language, a hardware design simulation, and/or other such similar mode(s) of expression). Alternatively or additionally, some or all of the logical expression may be manifested as a Verilog-type hardware description or other circuitry model before physical implementation in hardware, especially for basic operations or timing-critical applications. Those skilled in the art will recognize how to obtain, configure, and optimize suitable transmission or computational elements, material supplies, actuators, or other common structures in light of these teachings.

The foregoing detailed description has set forth various embodiments of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, or examples can be implemented, individually and/or collectively, by a wide range of hardware, software, firmware, or virtually any combination thereof. In one embodiment, several portions of the subject matter described herein may be implemented via Application Specific Integrated Circuits (ASICs), Field Programmable Gate Arrays (FPGAs), digital signal processors (DSPs), or other integrated formats. However, those skilled in the art will recognize that some aspects of the embodiments disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. In addition, those skilled in the art will appreciate that the mechanisms of the subject matter described herein are capable of being distributed as a program product in a variety of forms, and that an illustrative embodiment of the subject matter described herein applies regardless of the particular type of signal bearing medium used to actually carry out the distribution. Examples of a signal bearing medium include, but are not limited to, the following: a recordable type medium such as a floppy disk, a hard disk drive, a Compact Disc (CD), a Digital Video Disk (DVD), a digital tape, a computer memory, etc.; and a transmission type medium such as a digital and/or an analog communication medium (e.g., a fiber optic cable, a waveguide, a wired communications link, a wireless communication link (e.g., transmitter, receiver, transmission logic, reception logic, etc.), etc.).

In a general sense, those skilled in the art will recognize that the various embodiments described herein can be implemented, individually and/or collectively, by various types of electromechanical systems having a wide range of electrical components such as hardware, software, firmware, and/or virtually any combination thereof; and a wide range of components that may impart mechanical force or motion such as rigid bodies, spring or torsional bodies, hydraulics, electro-magnetically actuated devices, and/or virtually any combination thereof. Consequently, as used herein “electro-mechanical system” includes, but is not limited to, electrical circuitry operably coupled with a transducer (e.g., an actuator, a motor, a piezoelectric crystal, a Micro Electro Mechanical System (MEMS), etc.), electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of memory (e.g., random access, flash, read only, etc.)), electrical circuitry forming a communications device (e.g., a modem, communications switch, optical-electrical equipment, etc.), and/or any non-electrical analog thereto, such as optical or other analogs. Those skilled in the art will also appreciate that examples of electromechanical systems include but are not limited to a variety of consumer electronics systems, medical devices, as well as other systems such as motorized transport systems, factory automation systems, security systems, and/or communication/computing systems. Those skilled in the art will recognize that electro-mechanical as used herein is not necessarily limited to a system that has both electrical and mechanical actuation except as context may dictate otherwise.

In a general sense, those skilled in the art will recognize that the various aspects described herein which can be implemented, individually and/or collectively, by a wide range of hardware, software, firmware, and/or any combination thereof can be viewed as being composed of various types of “electrical circuitry.” Consequently, as used herein “electrical circuitry” includes, but is not limited to, electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of memory (e.g., random access, flash, read only, etc.)), and/or electrical circuitry forming a communications device (e.g., a modem, communications switch, optical-electrical equipment, etc.). Those having skill in the art will recognize that the subject matter described herein may be implemented in an analog or digital fashion or some combination thereof.

Those skilled in the art will recognize that at least a portion of the devices and/or processes described herein can be integrated into a data processing system. Those having skill in the art will recognize that a data processing system generally includes one or more of a system unit housing, a video display device, memory such as volatile or non-volatile memory, processors such as microprocessors or digital signal processors, computational entities such as operating systems, drivers, graphical user interfaces, and applications programs, one or more interaction devices (e.g., a touch pad, a touch screen, an antenna, etc.), and/or control systems including feedback loops and control motors (e.g., feedback for sensing position and/or velocity; control motors for moving and/or adjusting components and/or quantities). A data processing system may be implemented utilizing suitable commercially available components, such as those typically found in data computing/communication and/or network computing/communication systems.

Those skilled in the art wilt recognize that it is common within the art to implement devices and/or processes and/or systems, and thereafter use engineering and/or other practices to integrate such implemented devices and/or processes and/or systems into more comprehensive devices and/or processes and/or systems. That is, at least a portion of the devices and/or processes and/or systems described herein can be integrated into other devices and/or processes and/or systems via a reasonable amount of experimentation. Those having skill in the art will recognize that examples of such other devices and/or processes and/or systems might include—as appropriate to context and application—all or part of devices and/or processes and/or systems of (a) an air conveyance (e.g., an airplane, rocket, helicopter, etc.), (b) a ground conveyance (e.g., a car, truck, locomotive, tank, armored personnel carrier, etc.), (c) a building (e.g., a home, warehouse, office, etc.), (d) an appliance (e.g., a refrigerator, a washing machine, a dryer, etc.), (e) a communications system (e.g., a networked system, a telephone system, a Voice over IP system, etc.), (f) a business entity (e.g., an Internet Service Provider (ISP) entity such as Comcast Cable, Qwest, Southwestern Bell, etc.), or (g) a wired/wireless services entity (e.g., Sprint, Cingular, Nextet, etc.), etc.

In certain cases, use of a system or method may occur in a territory even if components are located outside the territory. For example, in a distributed computing context, use of a distributed computing system may occur in a territory even though parts of the system may be located outside of the territory (e.g., relay, server, processor, signal-bearing medium, transmitting computer, receiving computer, etc. located outside the territory).

A sale of a system or method may likewise occur in a territory even if components of the system or method are located and/or used outside the territory.

Further, implementation of at least part of a system for performing a method in one territory does not preclude use of the system in another territory.

All of the above U.S. patents, U.S. patent application publications, U.S. patent applications, foreign patents, foreign patent applications and non-patent publications referred to in this specification and/or listed in any Application Data Sheet, are incorporated herein by reference, to the extent not inconsistent herewith.

One skilled in the art will recognize that the herein described components (e.g., operations), devices, objects, and the discussion accompanying them are used as examples for the sake of conceptual clarity and that various configuration modifications are contemplated. Consequently, as used herein, the specific exemplars set forth and the accompanying discussion are intended to be representative of their more general classes. In general, use of any specific exemplar is intended to be representative of its class, and the non-inclusion of specific components (e.g., operations), devices, and objects should not be taken limiting.

Although user 118 is shown/described herein as a single illustrated figure, those skilled in the art will appreciate that user 118 may be representative of a human user, a robotic user (e.g., computational entity), and/or substantially any combination thereof (e.g., a user may be assisted by one or more robotic agents) unless context dictates otherwise. Those skilled in the art will appreciate that, in general, the same may be said of “sender” and/or other entity-oriented terms as such terms are used herein unless context dictates otherwise.

With respect to the use of substantially any plural and/or singular terms herein, those having skill in the art can translate from the plural to the singular and/or from the singular to the plural as is appropriate to the context and/or application. The various singular/plural permutations are not expressly set forth herein for sake of clarity.

The herein described subject matter sometimes illustrates different components contained within, or connected with, different other components. It is to be understood that such depicted architectures are merely exemplary, and that in fact many other architectures may be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled,” to each other to achieve the desired functionality, and any two components capable of being so associated can also be viewed as being “operably couplable,” to each other to achieve the desired functionality. Specific examples of operably couplable include but are not limited to physically mateable and/or physically interacting components, and/or wirelessly interactable, and/or wirelessly interacting components, and/or logically interacting, and/or logically interactable components.

In some instances, one or more components may be referred to herein as “configured to,” “configurable to,” “operable/operative to,” “adapted/adaptable,” “able to,” “conformable/conformed to,” etc. Those skilled in the art will recognize that “configured to” can generally encompass active-state components and/or inactive-state components and/or standby-state components, unless context requires otherwise.

While particular aspects of the present subject matter described herein have been shown and described, it will be apparent to those skilled in the art that, based upon the teachings herein, changes and modifications may be made without departing from the subject matter described herein and its broader aspects and, therefore, the appended claims are to encompass within their scope all such changes and modifications as are within the true spirit and scope of the subject matter described herein. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to claims containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that typically a disjunctive word and/or phrase presenting two or more alternative terms, whether in the description, claims, or drawings, should be understood to contemplate the possibilities of including one of the terms, either of the terms, or both terms unless context dictates otherwise. For example, the phrase “A or B” will be typically understood to include the possibilities of “A” or “B” or “A and B.”

With respect to the appended claims, those skilled in the art will appreciate that recited operations therein may generally be performed in any order. Also, although various operational flows are presented in a sequence(s), it should be understood that the various operations may be performed in other orders than those which are illustrated, or may be performed concurrently. Examples of such alternate orderings may include overlapping, interleaved, interrupted, reordered, incremental, preparatory, supplemental, simultaneous, reverse, or other variant orderings, unless context dictates otherwise. Furthermore, terms like “responsive to,” “related to,” or other past-tense adjectives are generally not intended to exclude such variants, unless context dictates otherwise.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1-72. (canceled)

73. A system, comprising:
a detector module; and
an alterer module configured to alter at least one artificial sensory experience at least partly based on detecting at least one indication of bioactive agent use by an individual.

74. The system of claim 73, wherein the detector module comprises:
an exhaled breath analysis detector module.

75. The system of claim 74, wherein the exhaled breath analysis detector module comprises:
a wireless exhaled breath analysis detector module.

76. The system of claim 73, wherein the detector module comprises:
a bioactive agent use detector module.

77. The system of claim 73, wherein the detector module comprises:
a medication-dispensing device detector module.

78. The system of claim 77, wherein the medication-dispensing device detector module comprises:
a prescription medication-dispensing device detector module.

79. The system of claim 73, wherein the detector module comprises:
a bioactive agent detector module.

80. The system of claim 73, wherein the detector module comprises:
an unregulated bioactive agent detector module.

81. The system of claim 80, wherein the unregulated bioactive agent detector module comprises:
an exhaled bioactive agent detector module.

82. The system of claim 73, wherein the detector module comprises:
a recreational bioactive agent detector module.

83. The system of claim 73, wherein the detector module comprises:
a transducer device detector module.

84. The system of claim 73, wherein the detector module comprises:
an alcohol detector module.

85. The system of claim 73, wherein the alterer module configured to alter at least one artificial sensory experience at least partly based on detecting at least one indication of bioactive agent use by an individual comprises:
an access alterer module.

86. The system of claim 85, wherein the access alterer module comprises:
a restricter module.

87. The system of claim 85, wherein the access alterer module comprises:
a granter module.

88. The system of claim 73, wherein the alterer module configured to alter at least one artificial sensory experience at least partly based on detecting at least one indication of bioactive agent use by an individual comprises:
a scene alterer module.

89. The system of claim 88, wherein the scene alterer module comprises:
a color scheme alterer module.

90. The system of claim 88, wherein the scene alterer module comprises:
a text alterer module.

91. The system of claim 73, wherein the alterer module configured to alter at least one artificial sensory experience at least partly based on detecting at least one indication of bioactive agent use by an individual comprises:
a sound alterer module.

92. The system of claim 91, wherein the sound alterer module comprises:
a music alterer module.

93. The system of claim 91, wherein the sound alterer module comprises:
an ambient sound alterer module.

94. The system of claim 91, wherein the sound alterer module comprises:
a voice alterer module.

95. The system of claim 73, wherein the alterer module configured to alter at least one artificial sensory experience at least partly based on detecting at least one indication of bioactive agent use by an individual comprises:
an efficacy alterer module.

96. The system of claim 73, wherein the alterer module configured to alter at least one artificial sensory experience at least partly based on detecting at least one indication of bioactive agent use by an individual comprises:
a side effect reduction alterer module.

97. The system of claim 73, wherein the alterer module configured to alter at least one artificial sensory experience at least partly based on detecting at least one indication of bioactive agent use by an individual comprises:
an adder module.

98. The system of claim 73, wherein the alterer module configured to alter at least one artificial sensory experience at least partly based on detecting at least one indication of bioactive agent use by an individual comprises:
a deleter module.

99. The system of claim 73, wherein the alterer module configured to alter at least one artificial sensory experience at least partly based on detecting at least one indication of bioactive agent use by an individual comprises:
a sensate alterer module.

100. The system of claim 99, wherein the sensate alterer module comprises:
a stimulus alterer module.

101. The system of claim 73, wherein the alterer module configured to alter at least one artificial sensory experience at least partly based on detecting at least one indication of bioactive agent use by an individual comprises:
a mobile device alterer module.
